(19) United States
(12) Patent Application Publication
(10) Pub. No.: US 2012/0174241 A1
(43) Pub. Date: Jul. 5, 2012

(54) Mouse model for diagnosis of T cell acute lymphoblastic leukemia and for screening of therapeutic agents, and methods of use therefor

(76) Inventors: Ludovic Deriano, New York, NY (US); David B. Roth, New York, NY (US)

(21) Appl. No.: 13/066,750

(22) Filed: Apr. 22, 2011

Related U.S. Application Data
(60) Provisional application No. 61/327,084, filed on Apr. 22, 2010.

(51) Int. Cl. A01K 67/027; C40B 30/04; G01N 21/64; G01N 33/53; C12N 5/10

(52) U.S. Cl. 800/10; 435/7.23; 435/354; 435/6.11; 506/9

(57) ABSTRACT

The invention provides mutant or transgenic animals and cells derived from the mutant or transgenic animals, and particularly a transgenic mouse, that is useful, among other things, for the study, prognosis and diagnosis of hematological malignancies, including T-cell acute lymphoblastic leukemia (T-ALL). Methods are provided for using the mouse model or mutant animal cells to assist in the discovery and identification of genes that may promote lymphomagenesis, to prognose and diagnose disease, and to screen for potential therapeutic agents or drugs.

### CROSS-REFERENCE TO RELATED APPLICATIONS

The present application is a non-provisional application claiming the priority of copending provisional application Ser. No. 61/327,084, filed Apr. 22, 2010, the disclosure of which is incorporated by reference herein in its entirety. Applicants claim the benefits of this application under 35 U.S.C. §119 (e).

### GOVERNMENT SUPPORT

The research leading to the present inventions was funded in part by Grant No. CA104588R from the National Institutes of Health. The government has certain rights in the invention.

### FIELD OF THE INVENTION

The present invention relates to mutant or transgenic animals and cells derived from the mutant or transgenic animals, and particularly to a transgenic mouse that is useful, among other things, for the study, prognosis and diagnosis of T-cell acute lymphoblastic leukemia (T-ALL). The invention relates to the use of the mouse model or mutant animal cells to assist in the discovery and identification of genes that may promote lymphomagenesis, to prognose and diagnose disease, and to screen for potential therapeutic agents, drugs, and the like.

### BACKGROUND OF THE INVENTION

Human T-cell leukemias can arise from oncogenes activated by specific chromosomal translocations involving T-cell receptor genes. In particular, T-cell acute lymphoblastic leukemia is a malignant disease of thymocytes, accounting for about 10% to about 15% of pediatric and about 25% of adult acute lymphoblastic leukemia cases. Although some therapies are available for T-cell acute lymphoblastic leukemia, they are not as effective as desired.

Few mouse models for the human disease T cell acute lymphoblastic leukemia (T-ALL) exist. The two most relevant are one in which a pair of activated oncogenes was put into a transgenic mouse (Aplan et al. An scl gene product lacking the transactivation domain induces bony abnormalities and cooperates with LMO1 to generate T-cell malignancies in transgenic mice. EMBO J (1997) vol. 16 (9) pp. 2408-19) thereby short-circuiting the normal process of incremental acquisition of genomic changes that activate oncogenes and delete tumor suppressor genes. Thus, these mice are not a realistic model. The second is a triple mutant mouse lacking telomerase, p53, and ATM. These mice display very high levels of genomic instability (Maser et al. Chromosomally unstable mouse tumours have genomic alterations similar to diverse human cancers. Nature (2007) vol. 447 (7147) pp. 966-71), yet lymphomas develop only after a substantial lag period (30 weeks) and the phenotype is incompletely penetrant (only 70% of the mice develop lymphomas). Furthermore, there is no evidence that telomerase is inactivated in human T-ALL.

Lymphocyte development is critically dependent upon V(D)J recombination of immunoglobulin (Ig) and T cell receptor (Tcr) genes, which generates a tremendous variety of antigen receptors. This process is regulated at multiple levels, but misrepair of DNA double-strand breaks (DSBs) generated by the V(D)J recombinases (the recombination activating gene (RAG) 1 and 2 encoded RAG1 and RAG2 proteins) is implicated in a variety of lymphoid malignancies in humans (1) and in mice (2-6).

Non-homologous end joining (NHEJ)-deficient mice cannot complete V(D)J recombination and are thus immunodeficient (41) although they can repair other types of DSBs through error-prone alternative NHEJ and other pathways. Despite the ongoing generation of progenitor lymphocytes with unrepaired, RAG-generated DSBs, NHEJ-deficient mice are not predisposed to lymphoma, presumably because cells bearing unrepaired DSBs are eliminated by p53-mediated apoptosis. In support of this contention, NHEJ/p53 doubly deficient mice rapidly develop RAG-dependent, pro-B cell lymphomas characterized by recurrent chromosome translocations involving the Ig loci, mediated by alternative NHEJ, and gene amplification (4-6, 41). Lymphomas and aberrant recombination events (including interlocus recombination, in which recombination occurs between different antigen receptor loci) have also been reported in animals deficient for the DNA damage response factors such as MRE11, NBS1 and ataxia telangiectasia mutated (ATM) (2,3,27, 28,31).

RAG mutations can cause specific defects in the joining stage of V(D)J recombination (12,13,16). RAG2 is of particular interest in this regard: the “core” domain (murine amino acids 1-383) is sufficient for recombination of artificial substrates in vitro and some antigen receptor loci in vivo (14,15,42), while a “dispensable” C-terminus (amino acids 384-526) contains a cell cycle-regulated protein degradation signal, a PHD domain that interacts with tri-methylated histone H3 and an acidic region capable of interacting with histones (14). Although the biological functions of these aforementioned elements remain unclear, the RAG2 C-terminus clearly plays important roles in vivo: core Rag2 knock-in (Rag2^{c/c}) mice (having only the core region amino acids 1-383 and lacking the remainder of the RAG2 protein by Cre-loxP-mediated genetic knock-in) show diminished V to DJ joining at Ig and Tcr loci (15,42) and aberrant re-insertion of excised signal end-containing DNA fragments (43). Loss of the RAG2 C-terminus impairs joining of artificial substrates (44), increases levels of DSBs (44) that persist through the cell cycle (18), and increases accessibility of the broken DNA ends to alternative NHEJ (12,19). Despite these defects, Rag2^{c/c} mice have not been reported to be lymphoma-prone.

Defects in DNA damage response factors, such as ATM or combined genetic deficiencies in nonhomologous end joining (NHEJ) and p53 proteins, predispose to V(D)J recombinase-initiated chromosome translocations, gene amplification, and lymphomagenesis (2,4-6,10,11,45). During V(D)J recombination, RAG1/RAG2 introduce DSBs at recombination signal sequences and maintain the DNA ends in a post-cleavage complex (46) that, at least in cultured cells, shepherds them to the NHEJ pathway for proper repair (12,13). Roles of the RAG proteins in preventing genomic instability, however, remain elusive.

There is clearly a need in the art for suitable and realistic animal models and cellular or system models for T-cell diseases, including T-cell leukemias, such as T-ALL.

The citation of references herein shall not be construed as an admission that such is prior art to the present invention.

### SUMMARY OF THE INVENTION

In a general aspect, the present invention relates In particular, the present invention relates to a double mutant mouse model that combines a C-terminal truncation of RAG2 (a component of the V(D)J recombinase) and loss of p53 to provide an animal model that develops T-cell lymphomas at a significant rate. 100% of homozygous double mutant mice develop T cell lymphomas within 15 weeks. This is in contrast to earlier models, which progress to T-cell disease slowly (on the order of 30 weeks) and in only a percentage of animals (about 70%), such as p53, ATM and telomerase triple mutant animal models.

The ensuing description demonstrates that the RAG2 C-terminus, although dispensable for recombination (14,15,42), is critical for maintaining genomic stability in developing lymphocytes and preventing lymphomagenesis. Thymocytes from mice homozygous for “core” Rag2, lacking the C-terminus (Rag2^{c/c} mice), show dramatic disruption of Tcrα/δ locus integrity. All Rag2^{c/c} p53^{-/-} mice rapidly develop aggressive thymic lymphomas bearing recurrent chromosomal translocations and gene amplification involving Tcrα/δ and Igh loci, reminiscent of lymphomas arising in an Atm^{-/-} background. In accordance with the present invention, novel role is provided for the C-terminus of RAG2, which serves as a tumor suppressor by maintaining genomic stability during antigen receptor gene assembly. The Rag2^{c/c}p53^{-/-} mouse provides a useful model for investigating the pathogenesis of T acute lymphoblastic leukemia (T-ALL), a common pediatric malignancy which often exhibits aberrant DNA rearrangements attributed to mistakes in V(D)J recombination.

The present invention provides RAG2/p53 double mutant animals, particularly mice, that rapidly develop T cell lymphoma (the most rapid and complete T lymphoma development yet known to have been reported in a mouse model to date), and further exhibit genome rearrangements characteristic of human T-ALL. These rearrangements occur by what appear to be the same mechanisms as lymphomagenic rearrangements in human T-ALL patients. The animals, including mice, of the present invention are useful for several purposes, including discovering genes important for driving lymphomagenesis which may be relevant in humans, for validating such genes as potential therapeutic targets, and developing and testing therapeutic agents in preclinical animal studies using the mouse model. The rapid onset of lymphomas, and their presence in 100% of animals, will greatly simplify determining effects of therapy.

In another aspect of the invention, a method for detecting and diagnosing T cell lymphoma (including T-ALL) in a patient is presented, said method comprising: a) isolating a cellular sample from the patient; and b) contacting a test substrate comprising the mouse model of the invention with said sample and c) examining said substrate for the extent of the development of lymphomagenesis.

The invention provides a transgenic mouse whose genome comprises a C-terminal truncation of RAG2 and the loss of p53, wherein said mouse is capable of the rapid development of T-cell lymphoma. In an aspect thereof, the C-terminal truncation of RAG2 results in RAG2 protein having only amino acids corresponding to amino acids 1-383 of mouse RAG2.

The present invention further provides a non-human transgenic animal model for hematologic malignancies caused by chromosomal translocations wherein the animal lacks p53 and its genome comprises a modified version of a gene encoding recombination activating gene 2 (RAG2) wherein the encoded RAG2 protein lacks a C-terminal region.

In an aspect of the animal model of the invention, the encoded RAG2 protein lacks amino acids corresponding to amino acids 384-526 in mouse RAG2. In an additional aspect, the RAG2 gene or encoding nucleic acid is deleted for nucleotides encoding amino acids corresponding to amino acids 384-526 in mouse RAG2. In a further aspect, the encoded RAG2 protein has only amino acids corresponding to amino acids 1-383 of mouse RAG2.

The animal model of the invention is applicable wherein the hematological malignancy is selected from T-cell acute lymphoblastic leukemia (T-ALL), B-cell acute lymphoblastic leukemia (B-ALL), diffuse large B-cell lymphoma (DLBCL) and Burkitt's lymphoma. In a particular aspect, the hematological malignancy is T-cell acute lymphoblastic leukemia (T-ALL).

The invention includes a non-human transgenic animal which is mutant for p53 and RAG2 whose genome comprises a modified version of a gene encoding p53 whereby p53 is lacking in the animal and whose genome also comprises a modified version of a gene encoding RAG2 wherein the encoded RAG2 protein lacks a C-terminal region.

In an aspect of the non-human transgenic animal, the encoded RAG2 protein lacks amino acids corresponding to amino acids 384-526 in mouse RAG2. In an additional aspect, the RAG2 gene or encoding nucleic acid is deleted for nucleotides encoding amino acids corresponding to amino acids 384-526 in mouse RAG2. In a further aspect, the encoded RAG2 protein has only amino acids corresponding to amino acids 1-383 of mouse RAG2.

In aspects of the animal model or the non-human transgenic animal of the present invention, the animal can be any animal species which is capable of genetic and transgenic modification. The animal may be selected, for example, from a non-human mammal (e.g., mouse, rat, rabbit, squirrel, hamster, rabbits, guinea pigs, pigs, micro-pigs, prairie dogs, baboons, squirrel monkeys and chimpanzees, etc), bird or an amphibian, in which one or more cells contain heterologous nucleic acid introduced by way of human intervention, such as by transgenic techniques well known in the art. For example, the animal can be selected from the group consisting of avian, bovine, canine, caprine, equine, feline, leporine, murine, ovine, porcine, non-human primate. The animal may be a mouse, dog or cat. The animal may be a rodent.

The invention includes a method of screening test drugs or agents that inhibit or suppress T-cell acute lymphoblastic leukemia, comprising: contacting or otherwise exposing the transgenic mouse of the present invention to a test drug or agent, wherein expression in T-lymphocytes rapidly induces T-cell acute lymphoblastic leukemia; comparing the leukemia in said transgenic mouse after contact or exposure to said test drug or agent relative to the leukemia of said mouse prior to contact or exposure with said test drug or agent; wherein suppression of the leukemia in said transgenic mouse after contact or exposure to said test drug or agent relative to the leukemia of said mouse prior to contact or exposure with said test drug or agent is indicative of a test drug or agent that suppresses T-cell acute lymphoblastic leukemia.

In an additional aspect of the invention a method is provided for screening a candidate compound for modulation of hematological malignancy comprising: (a) administering the candidate compound to the non-human transgenic animal of the present invention or to cells or a cell line derived from the non-human transgenic animal of the present invention; and (b) monitoring an effect of said compound on the non-human transgenic animal or on the cells or cell line.

In an aspect of the above method, monitoring the effect comprises detecting the levels of abnormal T cells in the animal or the cells of the cell line. In a further aspect, monitoring the effect comprises detecting the levels of chromosomal translocations in the animal, in the cells of the animal, or the cells of the cell line. In a still further aspect, monitoring the effect comprises detecting levels of genetic instability at the Tcrα/δ and Igh loci.

The invention provides a method of screening agents potentially useful for treating, preventing or inhibiting T-cell lymphoma, comprising: a) administering an agent to a first transgenic animal according to the present invention; b) observing the ability of the first transgenic animal to develop T-cell lymphoma; and c) comparing the ability of the first transgenic animal to develop T-cell lymphoma to the ability of a second transgenic animal according to the present invention to develop T-cell lymphoma, the agent not being administered to the second transgenic animal; wherein a decrease in development of T-cell lymphoma in the first transgenic animal indicates that the agent is potentially useful for treating, preventing or inhibiting T-cell lymphoma.

Cells, cell lines, or tissues derived from or isolated from the animals of the present invention are an additional inventive aspect thereof. The cells or cell lines may be useful in studies of lymphoma or in screening or assessing the effect of potential compounds, drugs or agents. Thus, the invention includes a cell or cell line derived from a non-human transgenic animal wherein the animal lacks p53 and its genome comprises a modified version of a gene encoding recombination activating gene 2 (RAG2) wherein the encoded RAG2 protein lacks a C-terminal region. The invention provides a cell or cell line derived from a non-human transgenic animal doubly mutant in lacking p53 and having mutant RAG2 protein which only comprises the core (corresponding to amino acids 1-383 of mouse RAG2) of RAG2.

Other objects and advantages will become apparent to those skilled in the art from a review of the ensuing detailed description, which proceeds with reference to the following illustrative drawings, and the attendant claims.

### BRIEF DESCRIPTION OF THE DRAWINGS

FIG. 1A-1C. The C terminus of RAG2 is a tumour suppressor in developing thymocytes. A, Kaplan-Meier tumour-free survival analysis for cohorts of control (wild type, n=12; Rag2^{c/c}, n=19), p53^{-/-} (n=32) and Rag2^{c/c}cp53^{-/-} (n=25) mice. Animals were monitored for 50 weeks. The average age of death in weeks is shown for p53^{31 /31} (22.8 weeks) and Rag2^{c/c} p53^{-/-} (12.1 weeks) genotypes with the P value determined by a Wilcoxon rank sum test. B, Tumour spectrum observed for Rag2^{c/c} p53^{-/-} (n=25) and p53^{-/-} mice (n=27). All Rag2^{c/c} p53^{-/-} animals (n=25) showed enlarged thymus. p53^{-/-} animals showed either enlarged thymus and/or spleen (n=18) or other non-lymphoid tumour mass (n=9). C, Physical appearance of normal thymus (wild type) and thymic lymphoma (Rag2^{c/c} p53^{-/-}, arrow) of 3-month-old animals.

FIG. 2. Rag2^{c/c} p53^{-/-} thymic lymphomas display recurrent translocations involving chromosomes that harbour antigen-receptor loci. Representative images of spectral karyotyping (1790T and 1745T) and G-bandkaryotyping (1779T) analysis of three Rag2^{c/c} p53^{-/-} T cell lymphomas. Metaphase number analysed and translocations for each tumour sample are listed in the table. All three tumours harbour clonal translocations involving chromosomes that carry Tcr (chromosome 14, Tcrα/δ; chromosome 6, Tcrβ) and/or Ig (chromosome 12, Igh; chromosome 6, Igκ; chromosome 16. Igλ) loci.

FIG. 3A-3D. Rag2^{c/c} p53^{-/-} thymocytes display Tcrα/δ- and Igh-associated genomic instability. A, Top panel: schematic of the Tcrα/δ locus, with positions of the BACs used for generation of DNA FISH probes indicated. Bottom panels: representative metaphases from two Rag2^{c/c} p53^{-/-} thymic lymphomas using the Tcrα/δ V BAC probe (red signal) combined with chromosome 14 paint (green signal, top row) or with the Tcrα/δ C BAC probe (green signal, bottom row). Arrows point to the amplification of the Tcrα/δ V region, arrowheads point to the translocated chromosome 14. B, Top panel: schematic of the Igh locus, with positions of the BACs used for generation of DNA FISH probes indicated. Bottom panels: representative metaphases from the same two Rag2^{c/c} p53^{-/-} thymic lymphomas using the Igh C BAC probe (red signal) combined with chromosome 12 paint (green signal, top row) or with the Igh V BAC probe (green signal, bottom row). Combination of chromosome 12 (red) and chromosome 14 (green) paints is shown for both tumours in black boxes. Arrowheads point to the translocated chromosome 12. C, Examples of confocal sections of three-dimensional Tcrα/δ DNA FISH on freshly isolated wild-type (top row) or Rag2^{c/c} (bottom rows) double-positive thymocytes. Tcrα/δ V (green) and C (red) BAC probes were used. Scale bar, 1 mm. D. Representative experiment showing the frequency at which Tcrα/δ V and/or Tcrα/δ C signals are lost in wild-type (WT), p53^{-/-} and Rag2^{c/c} thymocytes (n.200; see FIG. 15 for additional experiments and statistical analysis).

FIG. 4A-4C. The C terminus of RAG2 stabilizes the RAG post-cleavage complex. A, Biochemical end-release assay. Purified glutathione S-transferase (GST)-tagged core RAG1 and non-tagged RAG2 (full length or core) proteins (yellow circles) cleave a 500 base pair (bp) DNA substrate at 37° C. Post-cleavage signal end complexes are thermally challenged at increasing temperatures to force the release of signal ends, which are detected after electrophoresis and gel staining. B, Representative gel for end-release assays. Numbers above each lane indicate the temperatures (in degrees Celsius) the reactions were heated to before electrophoresis. CE, coding ends; SC, single cleavages; PK, samples treated with proteinase K and SDS. C, Quantification of signal end release, measured as the combined amount of signal ends divided by the signal from the total amount of DNA in the lane, from six experiments using two different protein preparations (*P<0.05, Student's t-test).

FIG. 5A and 5B. T cell development in Rag2^{c/c} and Rag1^{c/c} knock-in mice. Thymocytes were stained with APC-Cy7-anti-CD4, PE-Cy7-anti-CD8, APC-anti-CD25 and PE-anti-CD44. A, Upper, representative FACScan profile of live thymic lymphocytes from the indicated genotypes analyzed for the surface expression of CD4 and CD8. Lower, Percentages indicated are the mean and standard deviation of at least three repetitions of this experiment. B, Upper, representative FACScan profile of gated CD4⁻/CD8⁻ thymocytes from the indicated genotypes analyzed for the surface expression of CD25 and CD44. Lower, Percentages indicated are the mean and standard deviation of at least three repetitions of this experiment. (Complete analysis of the core-RAG2 and core-RAG1 knock-in mice have been previously published (15, 24).

FIG. 6. Flow cytometry analysis of Rag2^{c/c} p53^{-/-} thymic lymphomas. Representative FACScan profile of thymic lymphoma in Rag2^{c/c} Thymocytes derived from two lymphomas in Rag2^{c/c} p53^{-/-} mice and from a healthy wild type mouse were analyzed for the surface expression of CD4 and CD8. Plots with the FSC (x axis) versus SSC (y axis) indicate the large-sized lymphoma blasts in the tumors.

FIG. 7. PCR analysis of rearrangements at the Tcrβ locus in thymocytes and thymic lymphomas. PCR analysis of Dβ1 to Jβ1 and Dβ2 to Jβ2 rearrangements in WT, Rag2^{c/c}, Rag2^{-/-} thymocytes and Rag2^{c/c} p53^{-/-} thymic lymphomas was performed using primers specific for Dβ to Jβ rearrangements (upper panel), as previously reported (40). The bands marked by numbered arrows represent rearrangements of Dβ to one of the Jβ segments (except for pseudogene Jβ2.6*). Representative experiments are shown. The PCR primers, specific for the Dβ1 and Jβ1.6 and for the Dβ2 and Jβ2.7 segments, amplified respectively six Dβ1-Jβ1 and six Dβ2-Jβ2 rearrangements from wild-type and core Rag2 animals, reflecting the polyclonal nature of thymocytes in normal and Rag2^{c/c} mice. In contrast, tumor cells from the Rag2^{c/c} p53^{-/-} mice displayed generally one or two predominant rearrangements, indicating a clonal or oligoclonal origin.

FIG. 8. Tcrα/δ-associated genomic instability in Rag2^{c/c} p53^{-/-} thymic lymphomas. Top panel: schematic of the Tcrα/δ locus, with positions of the BACs used for generation of DNA FISH probes indicated. Bottom panels: representative metaphases from two Rag2^{c/c} p53^{-/-} thymic lymphomas (1779T and 1790T) analyzed by DNA FISH using the Tcrα/δ V BAC probe (green signal) in combination with the Tcrα/δ C BAC probe (red signal). Arrow heads point translocation events.

FIG. 9A and 9B. Absence of Tcrα/δ and Igh-associated genomic aberrations in p53^{-/-} thymic lymphomas. A, Top panel: schematic of the Tcrα/δ locus, with positions of the BACs used for generation of DNA FISH probes indicated. Bottom panel representative metaphases from one p53^{-/-} thymic lymphomas (6960T) analyzed by DNA FISH using the Tcrα/δ V BAC probe (red signal) combined with a chromosome 14 paint (green signal; left panel) or with the Tcrα/δ C BAC probe (green signal; right panel). B, Top panel: schematic of the Igh locus, with positions of the BACs used for generation of DNA FISH probes indicated. Bottom panels: representative metaphases from one p53^{-/-} thymic lymphoma (6960T) analyzed by DNA FISH using the Igh C BAC probe (red signal) combined with a chromosome 12 paint (green signal; left panel), or with the Igh V BAC probe (green signal; right panel).

FIG. 10A and 10B. array-CGH profile of chromosomes 14 and 12 from Rag2^{c/c} p53^{-/-} thymic lymphomas . Typical a-CGH profiles of chromosomes 14 (A) and 12 (B) from five Rag2^{c/c} p53^{-/-} thymic lymphomas (1348T, 1779T, 1780T, 2489T and 2805T). Red braces indicate gain of DNA material and green braces show loss of DNA material. The normalized hybridization signal (region mean in red) is plotted against the genomic location of the probes. Relative genomic positions of the Tcrα/δ locus (on chromosome 14) and Igh locus (on chromosome 12) are indicated.

FIG. 11A and 11B. array-CGH profile of chromosomes 14 and 12 from p53^{-/-} thymic lymphoma. Typical a-CGH profiles of chromosomes 14 (A) and 12 (B) from one p53^{-/-} thymic lymphomas (6960). The normalized hybridization signal (region mean in red) is plotted against the genomic location of the probes. Relative genomic positions of the Tcrα/δ locus (on chromosome 14) and Igh locus (on chromosome 12) are indicated.

FIG. 12A and 12B. The □ on □ core region of RAG2, but not RAG1, is a tumor suppressor in developing thymocytes. A, Kaplan-Meier tumor-free survival analysis for cohorts of control (WT, n=12 and Rag2^{c/c}, n=19), p53^{-/-} (n=32), Rag2^{c/c} p53^{-/-} (n=25) mice (as shown in FIG. 1) and Rag1^{c/c} p53^{-/-} (n=27) mice. Animals were monitored for 50 weeks. The average age of death in weeks is shown for p53^{-/-} (22.8 weeks), Rag2^{c/c} p53^{-/-} (12.1 weeks) and Rag1^{c/c} p53^{-/-} (18.7 weeks) genotypes. The P-value were determined by the Wilcoxon rank sum test; Rag1^{c/c} p53^{-/-} significantly different than p53^{-/-} (P(two-sided)=0.006) and Rag1^{c/c} p53^{-/-} highly significantly different than Rag2^{c/c} p53^{-/-} (P(two-sided)<0.0001). B, Pie chart showing the tumor spectrum observed for Rag2^{c/c} p53^{-/-} (n=25), p53^{-/-} (n=27) mice (as shown in FIG. 1) and Rag1^{c/c} p53^{-/-} (n=27) mice. All Rag2^{c/c} p53^{-/-} animals (n=25) showed enlarged thymus. Both p53^{-/-} and Rag1^{c/c} p53^{-/-} animals showed either enlarged thymus and/or spleen or other non lymphoid tumor mass with no statistical difference (Fisher's Exact Test, 2-Tail: p>0.5).

FIG. 13A and 13B. Absence of Tcrα/δ and Igh-associated genomic aberrations in Rag1^{c/c} p53^{-/-} thymic lymphomas. A, Top panel: schematic of the Tcralo locus, with positions of the BACs used for generation of DNA FISH probes indicated. Bottom panels: representative metaphases from two Rag2^{c/c} p53^{-/-} thymic lymphomas (8383T and 8411T) analyzed by DNA FISH using the Tcralo V BAC probe (red signal) combined with a chromosome 14 paint (green signal; top panels) or with the Tcrα/δ C BAC probe (green signal; lower panels). B, Top panel: schematic of the Igh locus, with positions of the BACs used for generation of DNA FISH probes indicated. Bottom panels: representative metaphases from two Rag2^{c/c} p53^{-/-} thymic lymphomas (8383T and 8411T) analyzed by DNA FISH using the Igh C BAC probe (red signal) combined with a chromosome 12 paint (green signal; top panels), or with the Igh V BAC probe (green signal; bottom panels).

FIG. 14A and 14B. array-CGH profile of chromosomes 14 and 12 from Rag1^{c/c} p53^{-/-} thymic lymphomas. Typical a-CGH profiles of chromosomes 14 (A) and 12 (B) from four Rag1^{c/c} p53^{-/-} thymic lymphomas (8315, 8333, 8383 and 8411). The normalized hybridization signal (region mean in red) is plotted against the genomic location of the probes. Relative genomic positions of the Tcrα/δ locus (on chromosome 14) and Igh locus (on chromosome 12) are indicated.

FIG. 15. Tcrα/δ locus integrity in wild type, Rag2^{c/c} and p53^{-/-} double positive thymocytes. Three experiments showing the frequency at which the Tcrα/δ V and/or the Tcrα/δ C signals are lost in wild-type, p53^{-/-} and Rag2^{c/c} thymocytes.

FIG. 16A and 16B. Atm^{-/-} thymic lymphomas display Tcrα/δ and Igh-associated genomic instability. A, Top panel: schematic of the Tcrα/δ locus, with positions of the BACs used for generation of DNA FISH probes indicated. Bottom panels: Representative metaphases from one Atm^{-/-} thymic lymphoma analyzed by DNA FISH using the Tcrα/δ V BAC probe (red signal) combined with a chromosome 14 paint (green signal; left panel) or with the Tcrα/δ C BAC probe (green signal; right panel). Arrows point to the amplification of the Tcrα/δ V region, arrow heads point to the translocated chromosome 14 and asterisks show a second translocation event of the Tcrα/δ C region on the chromosome that carries also the translocated distal end of chromosome 14. B, A typical array-CGH profile of chromosomes 14 and 12 from the same Atm^{-/-} tumor as in A. Red braces indicate gain of DNA material and green braces show loss of DNA material. The normalized hybridization signal (region mean in red) is plotted against the genomic location of the probes. Relative genomic positions of the Tcrα/δ locus (on chromosome 14) and Igh locus (on chromosome 12) are indicated.

FIG. 17A and 17B. Defective handling of V(D)J recombination intermediates in Rag2^{c/c} lymphocytes. A, Schematic showing the relative orientation of the Vκ6-23 to Jκ1 gene segments. RSS are shown as open triangle; arrows denote PCR primers. B, PCR analysis of Vκ6-23 to Jκ1 hybrid joints in splenocytes of indicated mouse genotypes using 300 ng of genomic DNA. PCR experiments were performed as previously described (3, 28).

FIG. 18. Table detailing genomic instability in Rag2^{c/c} p53^{-/-} thymic lymphomas. Analysis of Giemsa stained metaphase spreads prepared from 12 Rag2^{c/c} p53^{-/-} thymic lymphomas, two p53^{-/-} thymic lymphomas and two wild type thymi.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

The term “specific” may be used to refer to the situation in which one member of a specific binding pair will not show any significant binding to molecules other than its specific binding partner(s). The term is also applicable where e.g. an antigen binding domain is specific for a particular epitope which is carried by a number of antigens, in which case the specific binding member carrying the antigen binding domain will be able to bind to the various antigens carrying the epitope.

The term “comprise”generally used in the sense of include, that is to say permitting the presence of one or more features or components.

The term “consisting essentially of” refers to a product, particularly a peptide sequence, of a defined number of residues which is not covalently attached to a larger product. In the case of the peptide of the invention referred to above, those of skill in the art will appreciate that minor modifications to the N- or C- terminal of the peptide may however be contemplated, such as the chemical modification of the terminal to add a protecting group or the like, e.g. the amidation of the C-terminus.

The term “isolated” refers to the state in which specific binding members of the invention, or nucleic acid encoding such binding members will be, in accordance with the present invention. Members and nucleic acid will be free or substantially free of material with which they are naturally associated such as other polypeptides or nucleic acids with which they are found in their natural environment, or the environment in which they are prepared (e.g. cell culture) when such preparation is by recombinant DNA technology practised in vitro or in vivo. Members and nucleic acid may be formulated with diluents or adjuvants and still for practical purposes be isolated—for example the members will normally be mixed with gelatin or other carriers if used to coat microtitre plates for use in immunoassays, or will be mixed with pharmaceutically acceptable carriers or diluents when used in diagnosis or therapy.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term ‘agent’ means any molecule, including polypeptides, antibodies, polynucleotides, chemical compounds and small molecules. In particular the term agent includes compounds such as test compounds or drug candidate compounds.

The term ‘agonist’ refers to a ligand that stimulates the receptor the ligand binds to in the broadest sense.

The term ‘assay’ means any process used to measure a specific property of a compound. A ‘screening assay’ means a process used to characterize or select compounds based upon their activity from a collection of compounds.

The term ‘preventing’ or ‘prevention’ refers to a reduction in risk of acquiring or developing a disease or disorder (i.e., causing at least one of the clinical symptoms of the disease not to develop) in a subject that may be exposed to a disease-causing agent, or predisposed to the disease in advance of disease onset.

‘Therapeutically effective amount’ means that amount of a drug, compound, or pharmaceutical agent that will elicit the biological or medical response of a subject that is being sought by a medical doctor or other clinician. The phrase “therapeutically effective amount” is used herein to mean an amount sufficient to reduce by at least about 30 percent, more preferably by at least 50 percent, most preferably by at least 90 percent, a clinically significant reduction in a symptom or symptoms associated with a disease or disorder.

The term ‘treating’ or ‘treatment’ of any disease or infection refers, in one embodiment, to ameliorating the disease or infection (i.e., arresting the disease or growth of the infectious agent or bacteria or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or infection, either physically, (e.g., stabilization of a discernible symptom), physiologically, (e.g., stabilization of a physical parameter), or both. In a further embodiment, ‘treating’ or ‘treatment’ relates to slowing the progression of a disease.

The phrase “pharmaceutically acceptable” refers to molecular entities and compositions that are physiologically tolerable and do not typically produce an allergic or similar untoward reaction, such as gastric upset, dizziness and the like, when administered to a human.

As used herein, “pg” means picogram, “ng” means nanogram, “ug” or “μg” mean microgram, “mg” means milligram, “ul” or “μl” mean microliter, “ml” means milliliter, “l” means liter.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA.

A “clone” is a population of cells derived from a single cell or common ancestor by mitosis.

A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces -turns in the protein's structure.

Two amino acid sequences are “substantially homologous” when at least about 70% of the amino acid residues (preferably at least about 80%, and most preferably at least about 90 or 95%) are identical, or represent conservative substitutions.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567.

An “antibody combining site” is that structural portion of an antibody molecule comprised of heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Provided herein is a non-human animal model of hematological malignancies, particularly caused by chromosomal trasnslocation(s), wherein the cells of the animal do not express p53 and express an altered RAG2 protein which contains only RAG2 core and lacks the C-terminus of RAG2. The animal particularly provides an animal model of hematologic T-cell malignancy, particularly T-cell acute lymphoblastic leukemia (T-ALL).

Transgenic Animals

By a “transgene” is meant a nucleic acid sequence that is inserted by artifice into a cell and becomes a part of the genome of that cell and its progeny. Such a transgene may be (but is not necessarily) partly or entirely heterologous (e.g., derived from a different species) to the cell. The term “transgene” broadly refers to any nucleic acid that is introduced into an animal's genome, including but not limited to genes or DNA having sequences which are perhaps not normally present in the genome, genes which are present, but not normally transcribed and translated (“expressed”) in a given genome, or any other gene or DNA which one desires to introduce into the genome. This may include genes which may normally be present in the nontransgenic genome but which one desires to have altered in expression, or which one desires to introduce in an altered or variant form or in a different chromosomal location. A transgene can include one or more transcriptional regulatory sequences and any other nucleic acid, such as introns, that may be useful or necessary for optimal expression of a selected nucleic acid. A transgene can be as few as a couple of nucleotides long, but is preferably at least about 50, 100, 150, 200, 250, 300, 350, 400, or 500 nucleotides long or even longer and can be, e.g., an entire genome. A transgene can be coding or non-coding sequences, or a combination thereof. A transgene usually comprises a regulatory element that is capable of driving the expression of one or more transgenes under appropriate conditions. By “transgenic animal” is meant an animal comprising a transgene as described above. Transgenic animals are made by techniques that are well known in the art. The disclosed nucleic acids, in whole or in part, in any combination, can be transgenes as disclosed herein.

The disclosed transgenic animals can be any non-human animal, including a non-human mammal (e.g., mouse, rat, rabbit, squirrel, hamster, rabbits, guinea pigs, pigs, micro-pigs, prairie dogs, baboons, squirrel monkeys and chimpanzees, etc), bird or an amphibian, in which one or more cells contain heterologous nucleic acid introduced by way of human intervention, such as by transgenic techniques well known in the art. For example, the animal can be selected from the group consisting of avian, bovine, canine, caprine, equine, feline, leporine, murine, ovine, porcine, non-human primate. Thus, the animal can be a mouse, dog or cat. Thus, the animal can be a rodent.

Generally, the nucleic acid is introduced into the cell, directly or indirectly, by introduction into a precursor of the cell, such as by microinjection or by infection with a recombinant virus. The disclosed transgenic animals can also include the progeny of animals which had been directly manipulated or which were the original animal to receive one or more of the disclosed nucleic acids. This molecule may be integrated within a chromosome, or it may be extrachromosomally replicating DNA. For techniques related to the production of transgenic animals, see, inter alia, Hogan et al (1986) Manipulating the Mouse Embryo—A Laboratory Manual Cold Spring Harbor Laboratory, Cold Spring Harbor, N.Y., 1986).

Animals suitable for transgenic experiments can be obtained from standard commercial sources such as Charles River (Wilmington, Mass.), Taconic (Germantown, N.Y.), and Harlan Sprague Dawley (Indianapolis, Ind.). For example, if the transgenic animal is a mouse, many mouse strains are suitable, but C57BL/6 female mice can be used for embryo retrieval and transfer. C57BL/6 males can be used for mating and vasectomized C57BL/6 studs can be used to stimulate pseudopregnancy. Vasectomized mice and rats can be obtained from the supplier. Transgenic animals can be made by any known procedure, including microinjection methods, and embryonic stem cells methods. The procedures for manipulation of the rodent embryo and for microinjection of DNA are described in detail in Hogan et al., Manipulating the Mouse Embryo (Cold Spring Harbor Laboratory, Cold Spring Harbor, N.Y., 1986), the teachings of which are generally known and are incorporated herein.

Transgenic animals can be identified by analyzing their DNA. For this purpose, for example, when the transgenic animal is an animal with a tail, such as rodent, tail samples (1 to 2 cm) can be removed from three week old animals. DNA from these or other samples can then be prepared and analyzed, for example, by Southern blot, PCR, or slot blot to detect transgenic founder (F (0)) animals and their progeny (F (1) and F (2)). Thus, also provided are transgenic non-human animals that are progeny of crosses between a transgenic animal of the invention and a second animal. Transgenic animals can be bred with other transgenic animals, where the two transgenic animals were generated using different transgenes, to test the effect of one gene product on another gene product or to test the combined effects of two gene products.

The disclosed non-human animal and methods of making same obviate the need to immunocomprimise the animal. Thus, in some aspects, the disclosed non-human animal is not immunocompromised. Thus, in some aspects, the disclosed non-human animal is not a nude mouse.

The herein disclosed non-human animal models can comprise lymphoma, including T-cell lymphoma, particularly T-ALL. The animals can have an excess of white blood cells and may have T-cells and/or other blood cells with translocations. In particular the cells of the animals of the invention may have genomic instability at the Tcrα/δ and Igh loci, may contain clonal translocations and may contain chromosome 12/14 translocations.

The lymphomas generated in the disclosed model can show genomic instability at the Tcrα/δ and Igh loci, may contain clonal translocations and may contain chromosome 12/14 translocations.

Expression of the mutant RAG2 polypeptide, particularly the ‘core’ RAG2 polypeptide, and the lack of p53 expression or activity, in the disclosed non-human animal can be inducible or controllable. Thus, for example but not by way of limitation, the cells of the non-human animal can comprise a first and second polynucleotide, wherein the first polynucleotide comprises a nucleic acid sequence encoding a mutant RAG2 polypeptide, particularly the ‘core’ RAG2 polypeptide, operably linked to an expression control sequence (such as a immune-cell specific expression control sequence), and wherein the second polynucleotide comprises a mutant p53 gene or a p53 gene operably linked to a transcriptional termination signal, wherein the transcription'termination signal substantially prevents expression of the p53 polypeptide, wherein the non-human animal comprises T-cell lymphoma.

The RAG2 mutant polypeptide may lack the C-terminus, thus representing or having only an active ‘core’ RAG2 protein, via any means or genetic manipulation known in the art or available to the skilled artisan. This may be by deletion of the C-terminus encoding nucleic acids, by introduction of a premature stop codon or termination in the encoding nucleic acids, by replacement of the C-terminus encoding nucleic acids with distinct nucleic acids encoding a distinct and non-active sequence(s), by introduction of alternative amino acids or fusion to a distinct polypeptide or distinct amino acids without C-terminus activity or capability, etc.

The ‘core’ RAG2 gene in the animal of the invention may be generated by replacing a core only mutant for wild type RAG2 in the animal using homologous recombination, by knock-in technology, for instance using cre-loxP system, by germline correction using ES cells or cell lines, etc. The mutant gene or nucleic acid may utilize the sequence of the homologous species, for example mutant mouse sequence for wild type mouse sequence, or may utilize the gene or sequence from another distinct mammal or animal, including human, provided that the distinct gene or sequence replaces the ‘core’ activity of the wild type RAG2 in the transgenic animal. Exemplary and suitable RAG2 conrtuctsd are known in the art, including as utilized in the art-existing and available Rag2^{c/c} mutant animals utilized and referenced herein. Mammalian or animal RAG2 nucleic acid and amino acid sequences are known and available, including for example mouse RAG2 which is provided in Genbank sequences CAM20887.1 and AAI44857.

Nucleic acids encoding RAG and/or p53 can further comprise a nucleic acid sequence encoding a detection marker. This marker product is used to determine if the gene has been delivered to the cell and once delivered is being expressed. For example, the marker gene can be the E. Coli lacZ gene, which encodes β-galactosidase. The detection marker can be a fluorescent protein, such as green fluorescent protein. The marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes.

The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

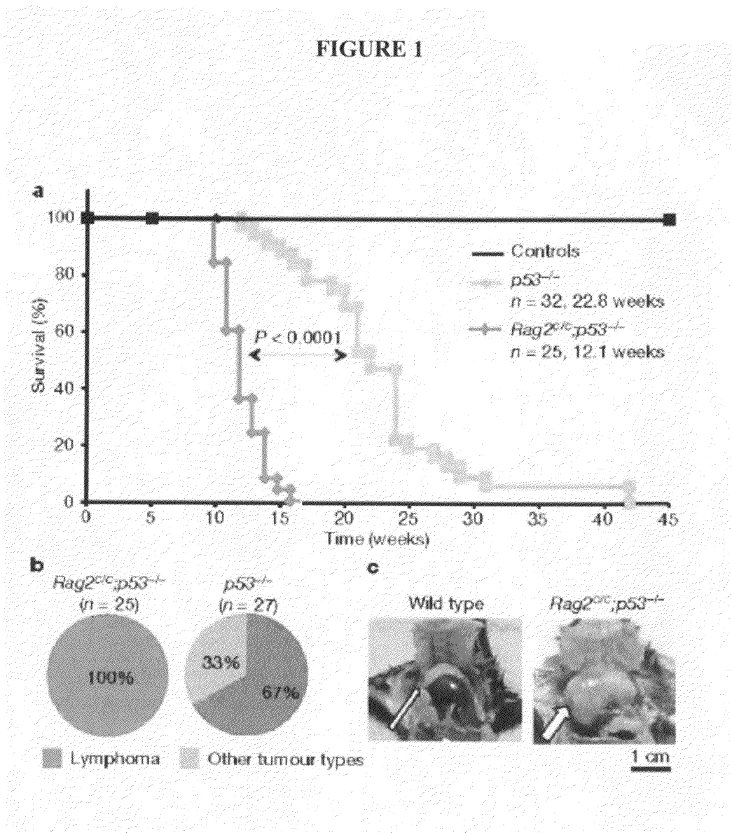

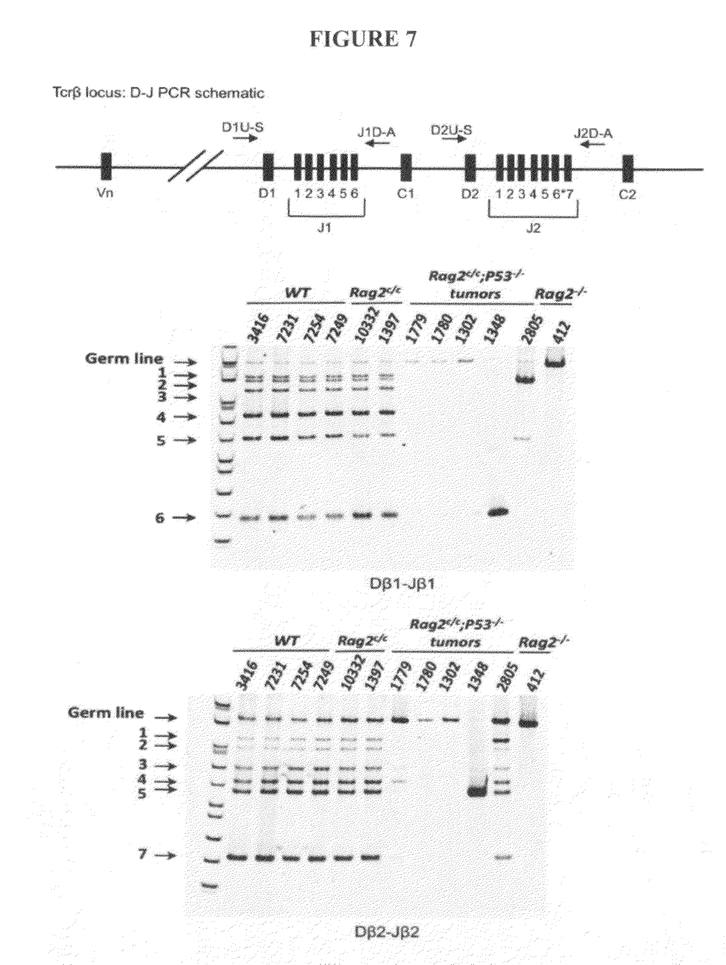

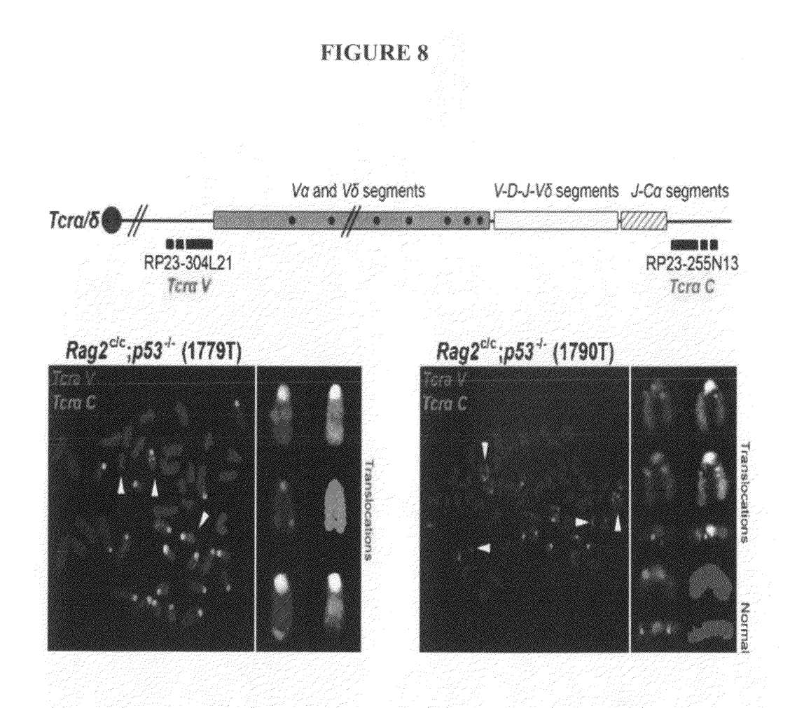

In accordance with the present invention, Rag2^{c/c} p53^{-/-} double mutant mice have been generated, in the belief that they might display genomic instability and lymphomagenesis, even in the context of intact NHEJ and early DNA damage response factors. Consistent with previous reports (15), our Rag2^{c/c} mice displayed partial developmental blocks in B and T lymphopoiesis because of a selective V-to-DJ rearrangement defect. Rag2^{c/c} animals, observed for up to one year, showed no obvious signs of tumorigenesis. In sharp contrast, 100% (n=25) of the animal model Rag p53 double mutants (Rag2^{c/c} p53^{-/-}), lacking p53 and the Rag C-terminal region, animals (mice) died within 16 weeks (mean survival=12.1 weeks) with aggressive thymic lymphomas (FIG. 1). Tumor cells were highly proliferative and invariably expressed cell surface CD4 and CD8, with little or no surface TCR (CD3E or TCR(3), indicating that these tumors originate from immature thymocytes. Thymic lymphomas appeared early: tumors with highly proliferating lymphoblasts were detected in 4- to 6-week-old Rag2^{c/c} p53^{-/-} thymi, but not in other organs, confirming their thymic origin. Tumor cells from the Rag2^{c/c} p53^{-/-} mice generally displayed only one or two predominant Dβ2-Jβ2 rearrangements, indicating a clonal or oligoclonal origin (FIG. 7).

It is well established that thymic lymphomas arising in p53^{-/-} mice lack clonal chromosome translocations, although aneuploidy is commonly observed (21,23). Given the striking ability of core RAG2 to accelerate lymphomagenesis, we examined genomic stability in lymphomas from Rag2^{c/c} p53^{-/-} mice, first by analysis of Giemsa stained metaphase spreads prepared from 12 thymic lymphomas (FIG. 18). As expected, wild-type thymocytes showed very low levels of abnormal metaphases (normal diploid metaphases: 97% to 100%).In contrast, p53^{-/-} tumors harbored a large array of cytogenetic aberrations (normal diploid metaphases: between 5.9% to 83.3%), including aneuploidy, chromosome breaks, and chromosome fusions (FIG. 18).

Although the particular RAG2 deletion mouse used herein has been created independently by two groups Liang, H. E. et al., The “dispensable” portion of RAG2 is necessary for efficient V-to-DJ rearrangement during B and T cell development. Immunity 17 (5), 639-651 (2002); Akamatsu et al. Deletion of the RAG2 C terminus leads to impaired lymphoid development in mice. Proceedings of the National Academy of Sciences (2003) vol. 100 (3) pp. 1209; and the p53-deficient mice were developed many years ago (Donehower et al. Mice deficient for p53 are developmentally normal but susceptible to spontaneous tumours. Nature (1992) vol. 356 (6366) pp. 215-21), it was not suspected that the RAG2 mutation would enhance lymphomagenesis until we had discovered a novel role for the RAG-2 C-terminus (Corneo et al, Nature), which explains why no papers have been published showing the combined RAG2/p53 mutation in the 8 years since the RAG2 core mouse was first published. We reasoned that combining a mutation which causes release of broken DNA ends might cause genomic instability.

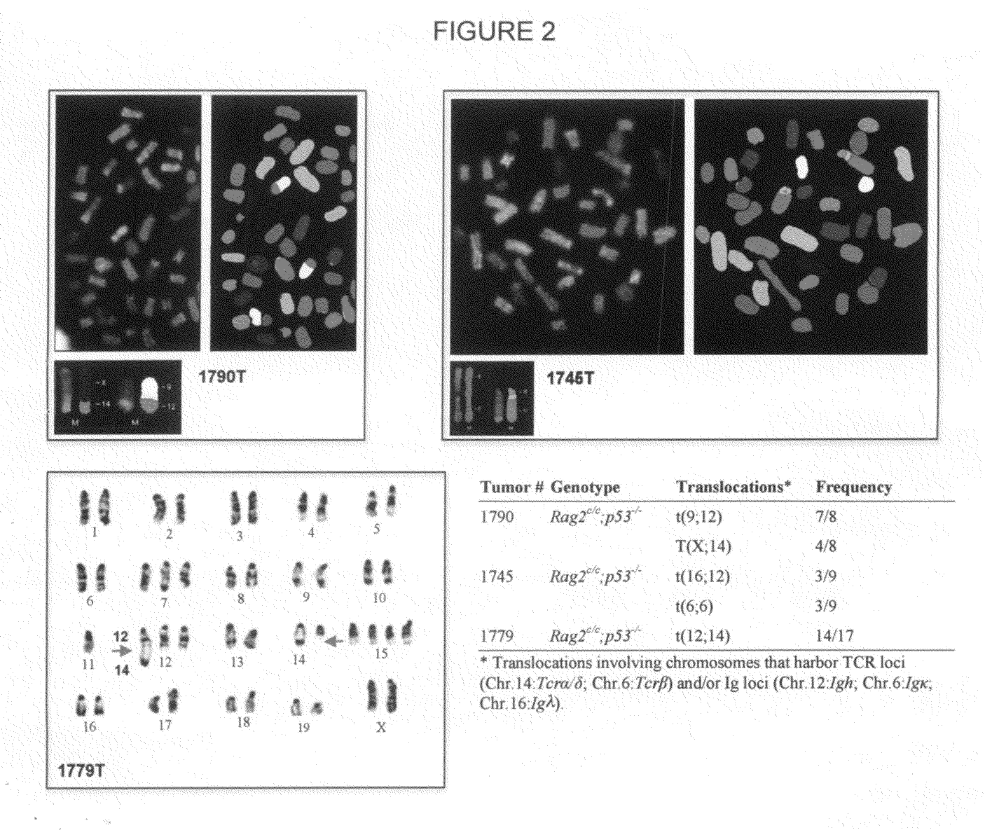

Detailed cytogenetic analyses were performed on three Rag2^{c/c} p53^{-/-} thymic lymphomas using spectral karyotyping (SKY) (tumors 1790T and 1745T) and G-band karyotyping (tumor 1779T) (FIG. 2). In contrast to p53^{-/-} lymphomas (21,23), recurrent translocations were observed involving chromosomes that harbor Tcr (Chr. 14 and 6) and Ig (Chr. 12, 6 and 16) loci, suggesting that these might have been initiated by RAG-generated breaks. All three lymphomas harbored translocations of the Igh locus-containing chromosome 12 and/or the Tcrα/δ locus-containing chromosome 14, loci which undergo rearrangement in thymocytes (22,48). Analysis of lymphoma 1779T revealed a C12;14 translocation (FIG. 2). These results suggest that Rag2^{c/c} p53^{-/-} T cell tumors harbor clonal translocations involving the Tcrα/δ and Igh loci, as seen in T cell lymphomas from ataxia-telangiectasia patients and Atm^{-/-} mice (8,10,49).

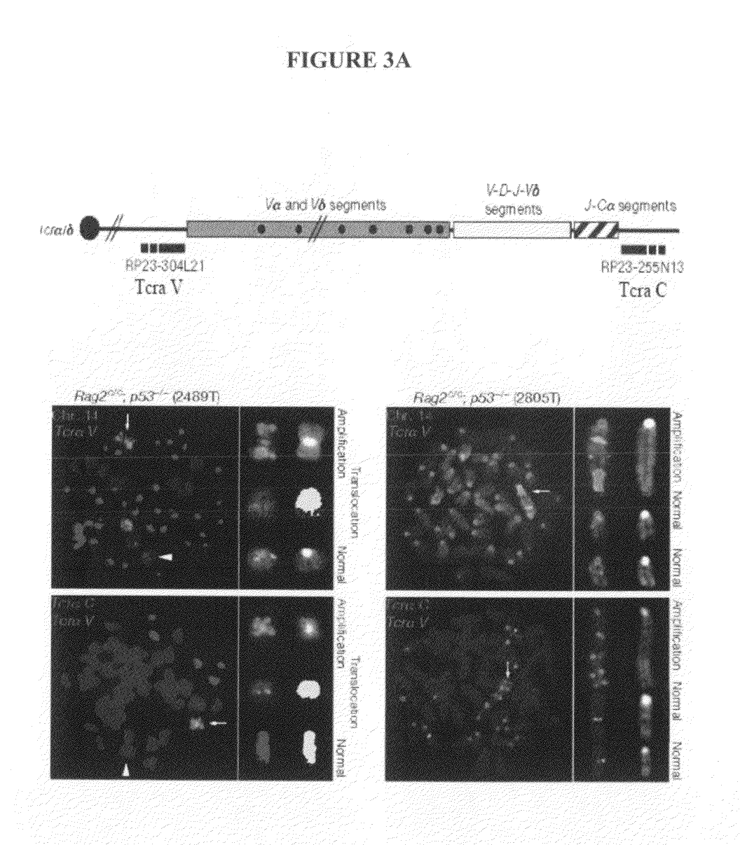

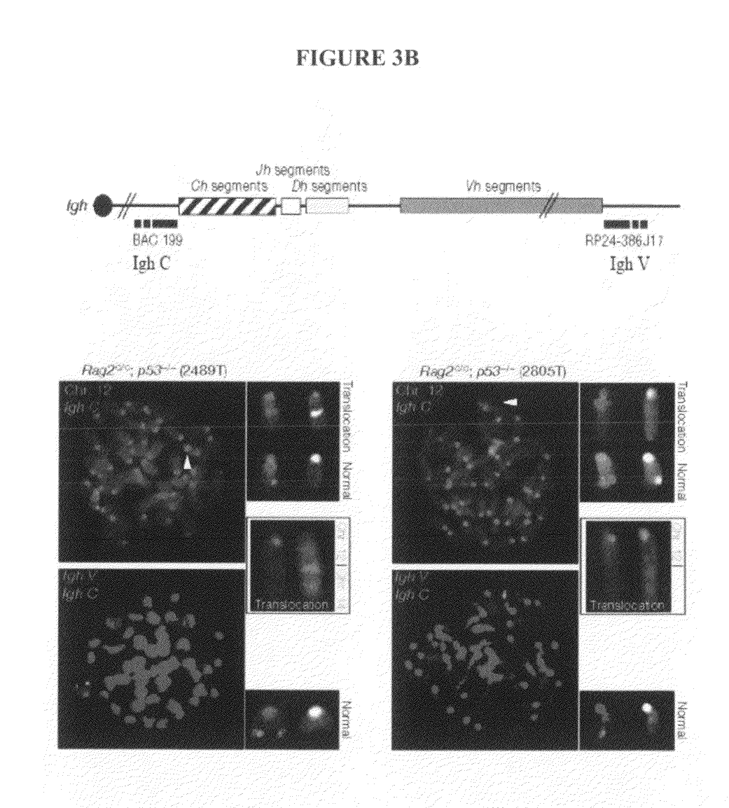

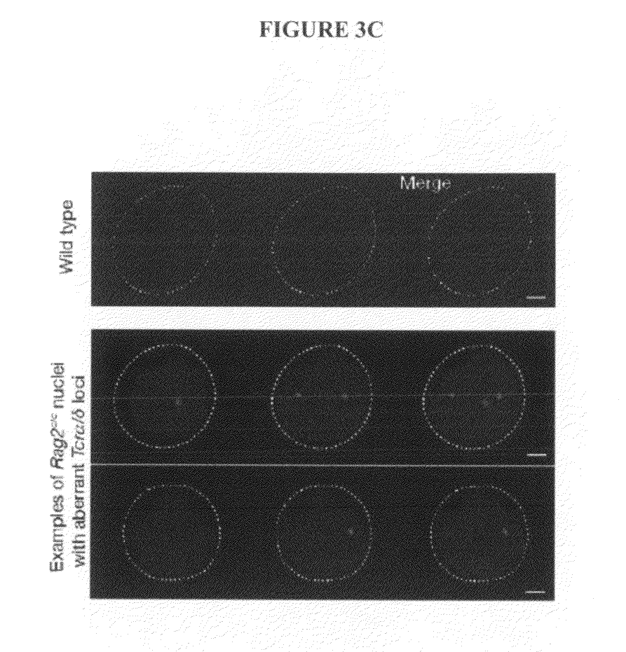

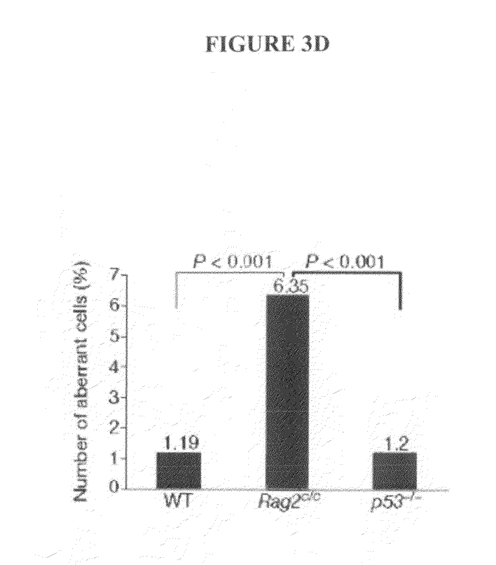

To confirm the involvement of the Tcrα/δ and Igh loci in chromosome translocation DNA-fluorescent in situ hybridization (DNA-FISH) analyses was performed on metaphase spreads from two Rag2^{c/c} p53^{-/-} thymic lymphomas (2489T and 2805T). Two BAC probes specific for the region upstream of the Tcrα/δ variable segments (Tcrα/δ V, centromeric of Tcrα/δ) and for the region downstream of the Tcrα constant segment (Tcrα/δ C, telomeric of Tcrα/δ) and a paint for chromosome 14 (FIG. 3A) were used in the analysis. In both tumors, breakpoints within the Tcrα/δ locus of one of the two chromosomes 14 resulted in amplification of the Tcrα/δ V region (FIG. 3A). The broken telomeric part of the chromosome (including Tcrα/δ C) was either translocated (2489T), or lost (2805T). DNA-FISH analysis of tumors 1790T and 1779T (FIG. 2) using Tcrα/δ C and V probes also confirmed translocation of chromosome 14 with breakpoints within the Tcrα/δ locus, although without obvious amplification (FIG. 7).

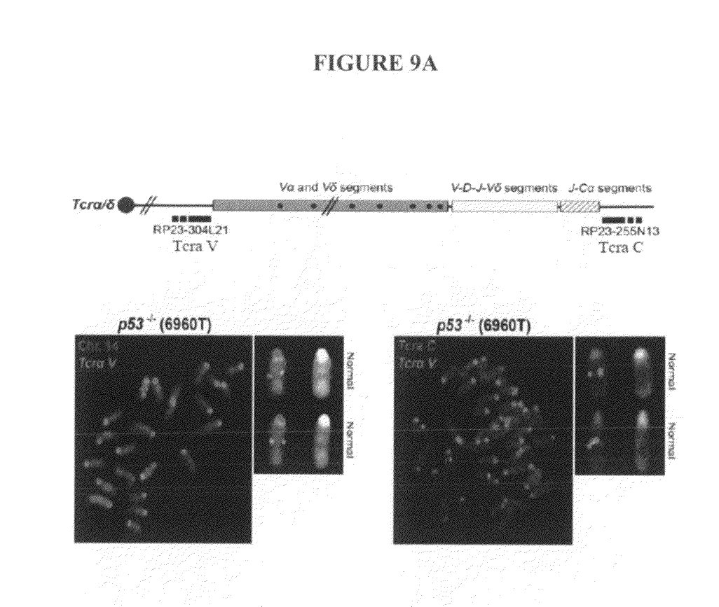

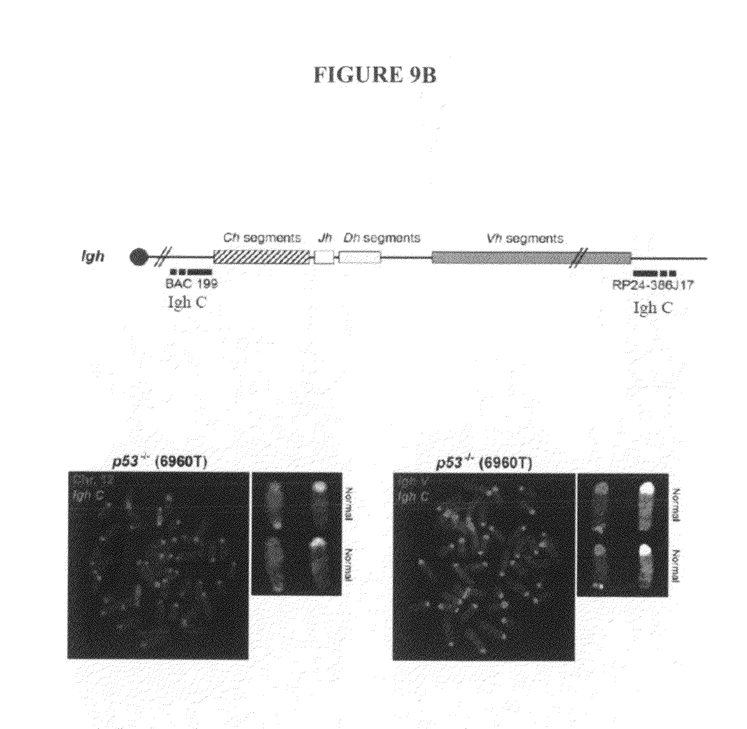

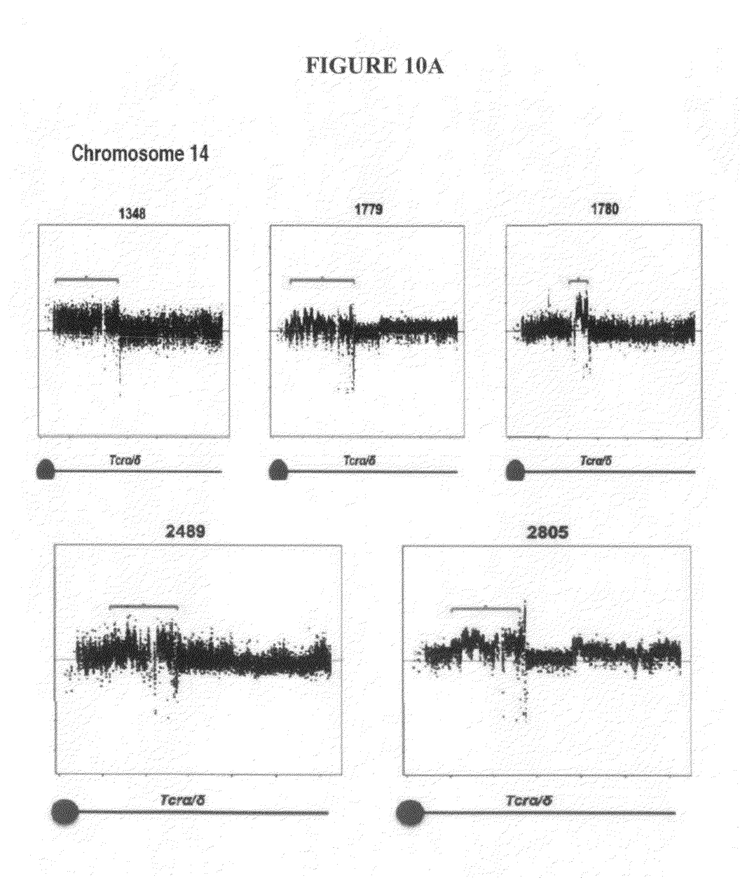

The status of the Igh locus in metaphase spreads was assessed from the two Rag2^{c/c} p53^{-/-} thymic lymphomas 2489T and 2805T. DNA-FISH was performed using BAC probes flanking the Igh locus (Igh C upstream of the constant segments (centromeric of Igh), and Igh V downstream of the variable segments (telomeric end)) and a chromosome 12 paint (FIG. 3B). In both thymic lymphomas, one chromosome 12 showed translocation with another chromosome, with accompanying loss of the Igh C and V signals. The breakpoints induced by core RAG2 resulted in the loss of the telomeric end of the chromosome (including Igh n (FIG. 3B). The loss of the Igh C region is likely due to end degradation before fusion to the partner chromosome, as previously reported in Atm^{-/-} mouse T cells (8). Chromosome 12 and 14 paint analysis showed a C12;14 translocation in lymphoma 2489T (FIG. 3B). DNA-FISH in p53^{-/-} thymic lymphomas indicated that, in contrast to Rag2^{c/c} p53^{-/-} lymphomas, both Tcrα/δ and Igh loci were intact (FIG. 9), consistent with previous work showing that p53^{-/-} lymphomas lack translocations of Tcr- or Ig-bearing chromosomes (21). These data reveal a novel in vivo function for the C-terminus of RAG2, whose loss promotes genomic instability at the Tcrα/δ and Igh loci in p53-deficient thymocytes, resulting in clonal chromosome translocations and amplifications.

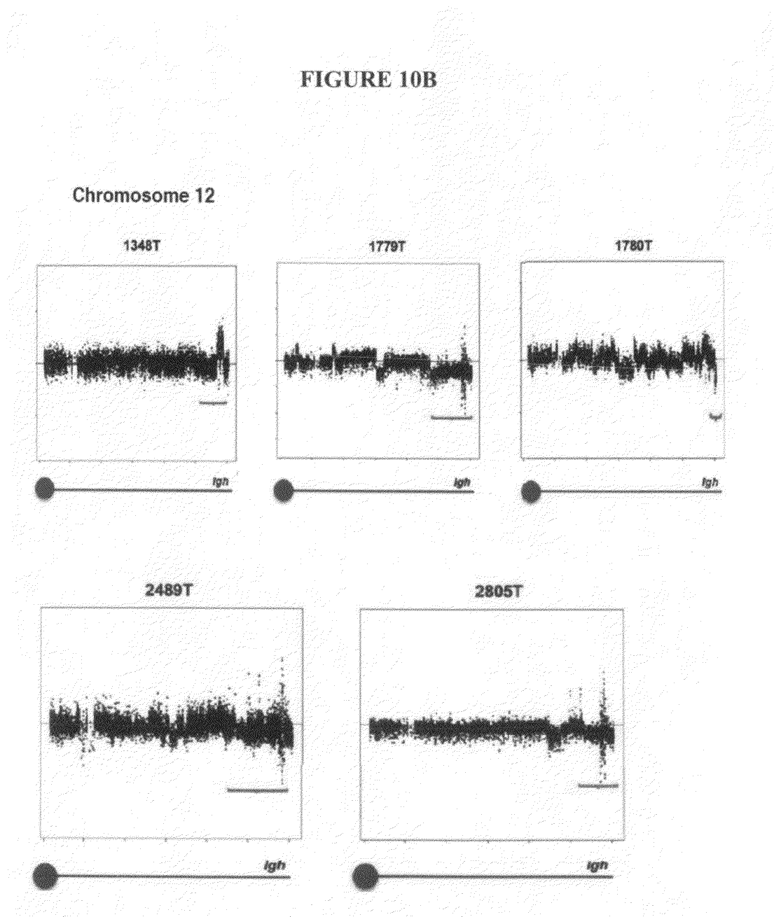

To examine genomic aberrations in these tumors in greater detail, array-based comparative genomic hybridization (array CGH or aCGH) analysis was performed on DNA from Rag2^{c/c} p53^{-/-} thymic lymphomas. Although only whole chromosome gains have been reported in p53^{-/-} thymic lymphomas (23), all three Rag2^{c/c} p53^{-/-} lymphomas examined showed substantial amplification of a common region on chromosome 14, centromeric of the Tcrα/δ^{locus}. These data are in agreement with the FISH analyses (FIG. 3A). Tumor samples also showed loss of a region within the Tcrα/δ locus, likely reflecting V(D)J recombination (FIG. 10). Amplification of a distal region of chromosome 12 centromeric of the Igh locus was observed in tumors 1348T and 1780T, and loss of the distal end of chromosome 12, telomeric of the Igh locus was seen in tumor 1780T. These results confirm that the recurrent chromosome translocations observed at Tcrα/δ and Igh loci in Rag2^{c/c} p53^{-/-} mice are often associated with amplification of genomic regions centromeric of the loci.

Amplification events associated with RAG-mediated breakage at antigen receptor loci suggests a “breakage-fusion-bridge” model (4,41) in which a translocation leads to formation of a dicentric chromosome, leading to repeated cycles of breakage and rejoining as the cells undergo division. Unlike NHEJ/p53 doubly-deficient animals, which develop pro-B lymphomas that contain a C12;15 translocation associated with co-amplification of the c-myc gene and Igh sequences (4-6,41), Rag2^{c/c} p53^{-/-} mice develop thymic lymphomas that contain chromosome translocations at Tcrα/δ (chr.14) and Igh loci (chr.12) often associated with genomic amplification. Atm^{-/-} mice are also predisposed to thymic lymphomas, all of which harbor chromosome 14 translocations that involve the Tcrα/δ locus, with mouse chromosome 12 as the translocation partner in approximately 50% of the cases (8,10,11).

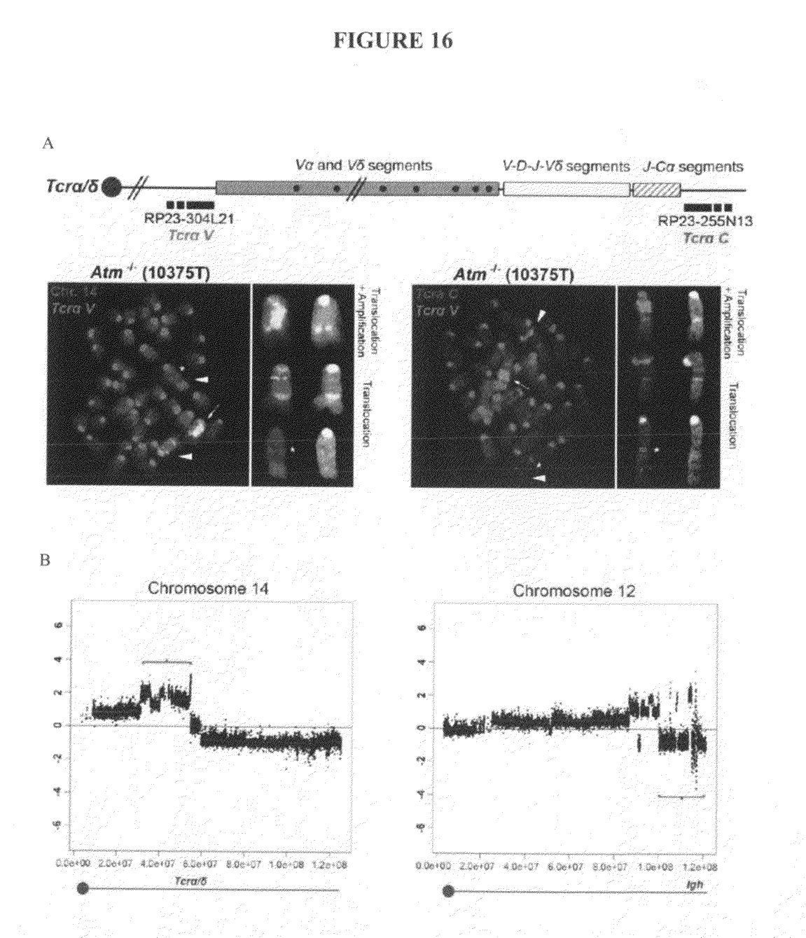

To determine whether Atm^{-/-} thymic lymphomas also harbor amplification of genomic regions close to the Tcrα/δ locus, we performed DNA-FISH analysis for Tcrα/δ and chromosome 14 on metaphase spreads from one Atm^{-/-} thymic lymphoma (FIG. 16A). Both chromosomes 14 had undergone translocations with breakpoints within the Tcrα/δ locus, and amplification of the Tcrα/δ V region on one allele.

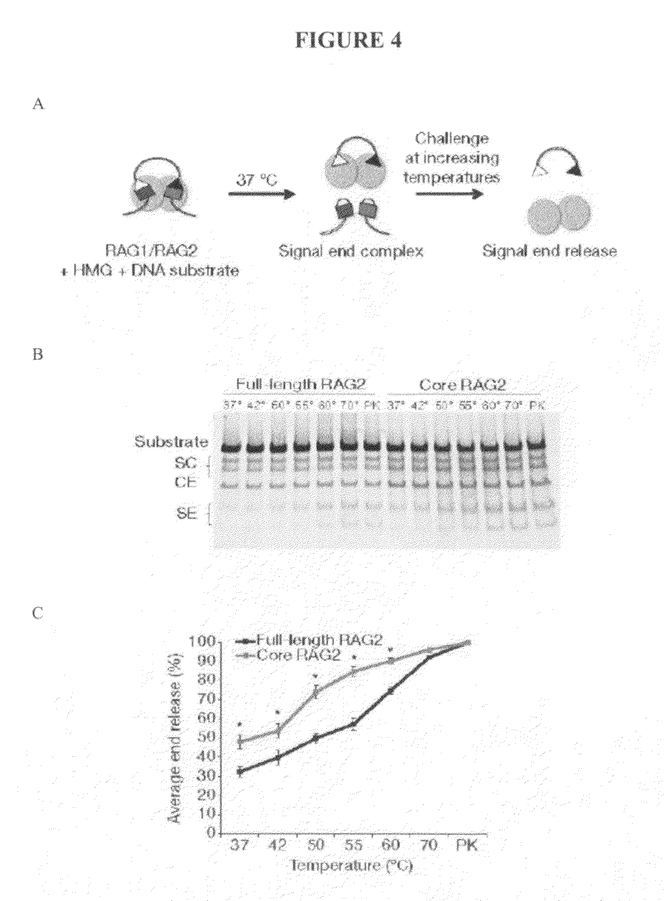

Core-RAG2 expressing animals are not predisposed to lymphomagenesis in a p53 wild-type background. Genomic instability was then determined in double positive (DP) thymocytes from Rag2^{c/c} mice using three-dimensional interphase DNA-FISH to examine the integrity of Tcrα/δ locus (FIG. 4). The two alleles appeared as two pairs of signals (Igh C and Igh V, mapping the two ends of the locus) in most (>98%) wild-type and p.53^{-/-} DP thymocytes, indicating that p53 deficiency alone does not disrupt the integrity of the Tcrα/δ locus, in agreement with previous studies (25). In contrast, Rag2^{c/c} DP thymocytes displayed a three- to five-fold increase in the number of aberrant cells showing loss of at least one signal. These results suggest that loss of the RAG2 C-terminus promotes genomic instability at the Tcrα/δ locus, a phenotype similar to that previously reported in Atm^{-/-} and 53bp1^{-/-} animals (9,25).

Together, these observations indicate that the RAG2 C-terminus enforces proper handling of RAG-generated DSBs, which, when mishandled, are lymphomagenic in the absence of p53. Based on these results it is proposed that the C-terminus of RAG2 is important for stabilizing the RAG post-cleavage complex, a hypothesis supported by increased alternative NHEJ in the presence of core RAG2 (12,19). This hypothesis was tested in vitro, and found that, compared with fill-length RAG2, purified core RAG-2 indeed destabilizes RAG/signal end complexes. The findings support a model in which premature release of DSBs allows ends to escape the normal joining mechanisms and persist, eventually being joined by alternative NHEJ, a joining pathway permissive for chromosome translocations (4,29) and amplification events (4). Abnormal persistence of RAG activity through the cell cycle resulting from impaired degradation of core RAG2 (18,30) would-amplify the dangers posed by unrepaired ends, which would be further augmented by the absence of the p53-dependent checkpoint, explaining the synergy between core RAG2 and p53-deficiency in lymphomagenesis.

The model is also supported by several striking and unexpected similarities between Rag2^{c/c} p53^{-/-} and Atm^{-/-} mice: both feature RAG-dependent genomic instability at the Tcrα/δ and Igh loci, with development of pro-T cell lymphomas bearing clonal translocations, including 12/14 translocations-and amplification involving these loci. These similarities may reflect common underlying mechanisms: like core RAG2, ATM stabilizes the RAG post-cleavage complex (3), and persistent RAG-induced DSBs are observed in ATM-deficient lymphocytes (2,3), presumably because ATM is responsible for activating p53-dependent cell cycle checkpoints and apoptosis. Together, these observations suggest that one way in which RAG2 serves as a tumor suppressor is to ensure proper handling of RAG-generated DNA ends.

The complete penetrance and rapid development of early thymic lymphomas displaying high levels of RAG-mediated genomic instability make the Rag2^{c/c} p53^{-/-} mice an attractive model for investigating the pathogenetic steps underlying lymphomagenesis. Given the characteristic chromosome translocations involving antigen receptor gene loci, Rag2^{c/c} p53^{-/-} mice provide an interesting model for investigating the pathogenesis of T acute lymphoblastic leukemia (T-ALL), a common pediatric malignancy which often exhibits aberrant DNA rearrangements attributed to mistakes in V(D)J recombination (44).

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### EXAMPLE

Misrepair of DNA double-strand breaks produced by the V(D)J recombinase (the RAG1/RAG2 proteins) at immunoglobulin (Ig) and T cell receptor (Tcr) loci has been implicated in pathogenesis of lymphoid malignancies in humans (1) and in mice (2-7). Defects in DNA damage response factors such as ataxia telangiectasia mutated (ATM) protein and combined deficiencies in classical non-homologous end joining and p53 predispose to RAG-initiated genomic rearrangements and lymphomagenesis (2-11). Although we showed previously that RAG1/RAG2 shepherd the broken DNA ends to classical nonhomologous end joining for proper repair (12,13), roles for the RAG proteins in preserving genomic stability remain poorly defined. Here we show that the RAG2 carboxy (C) terminus, although dispensable for recombination (14,15), is critical for maintaining genomic stability.

RAG2 protein has a “core” domain (amino acids 1-383) and a C-terminus, denoted “dispensable” (corresponding to amino acids 384-526). Since full-length RAG1 and RAG2 are insoluble at physiological salt concentrations, biochemical studies of RAG1 and RAG2 historically relied on truncated, catalytically active recombinant proteins, which are readily soluble. These core-RAG1 (amino acids 384-1008 of 1040 residues) and core-RAG2 (amino acids 1-383 of 527 residues) proteins were initially defined as the minimal regions of each protein essential for near-wild-type recombination activity on cotransfected extrachromosomal reporter substrates in nonlymphoid cell lines (reviewed in Fugmann, S. D and Schatz, D. G. (2000) Mol Cell 8:899-910). Core-RAG2 knock-in mice have been previously generated using a Cre-loxP strategy, replacing the full-length RAG2 coding sequence with core-RAG2, and retaining the 3′ untranslated region of RAG2, so as to closely preserve the endogenous regulation of RAG2 (Liang, HE et al (2002) Immunity 17:639-651).

We now demonstrate that hymocytes from ‘core’ Rag2 homozygotes (Rag2^{c/c} mice) show dramatic disruption of Tcrα/δ locus integrity. Furthermore, all Rag2^{c/c} p53^{-/-} mice, unlike Rag1^{c/c} p53^{-/-} and p53^{-/-} animals, rapidly develop thymic lymphomas bearing complex chromosomal translocations, amplifications and deletions involving the Tcrα/δ and Igh loci. These mice were generated because the V(D)J recombinase (RAG1 and RAG2) has been implicated in generating oncogenic DNA rearrangements in lymphomas, especially in ALL. It was previously reported that removing the C-terminus of RAG2 increases the frequency of aberrant rearrangements in cultured cells (Corneo et al. Rag mutations reveal robust alternative end joining. Nature (2007) vol. 449 (7161) pp. 483-486).

We also find these features in lymphomas from Atm“” mice. We show that, like ATM-deficiency (3), core RAG2 severely destabilizes the RAG post-cleavage complex. These results reveal a novel genome guardian role for RAG2 and suggest that similar ‘end release/end persistence’ mechanisms underlie genomic instability and lymphomagenesis in Rag2^{c/c} p53^{-/-} and Atm^{-/-} mice.

RAG mutations can cause specific defects in the joining stage of V(D)J recombination (12,13,16). The ‘dispensable’ RAG2 C terminus (murine amino acids 1-383) is of particular interest: loss of the RAG2C terminus impairs joining of substrates (17), increases levels of double-strand breaks (17) that persist through the cell cycle (18), and increases accessibility of the broken DNA ends to alternative non-homologous end joining(12,19). Despite these defects, Rag2^{c/c} mice are not lymphoma-prone.

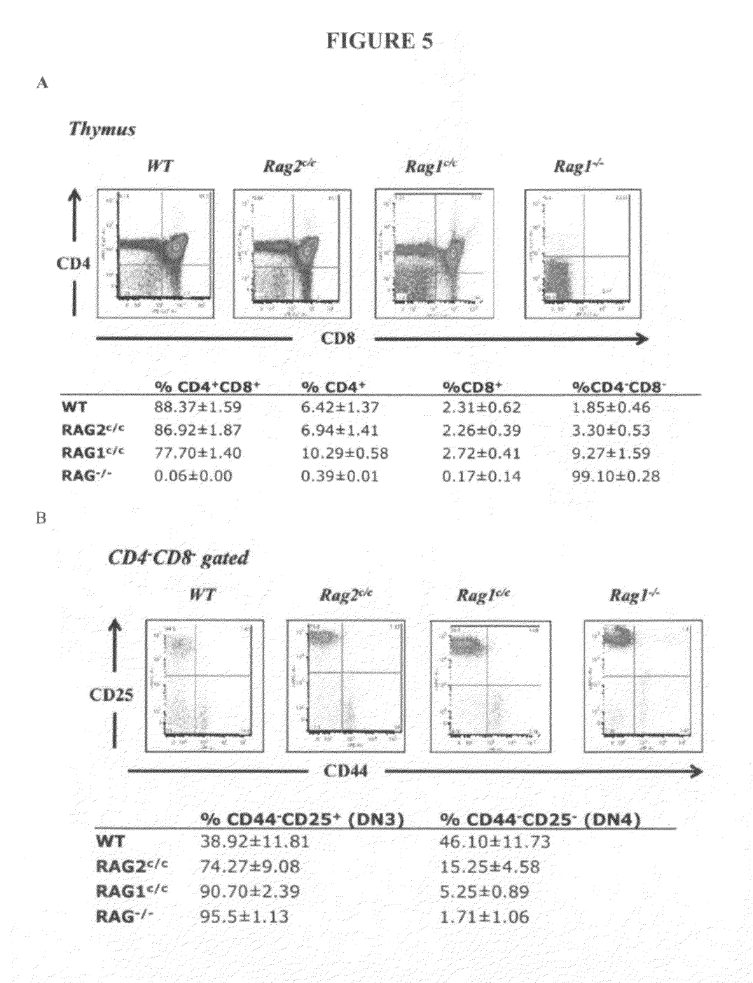

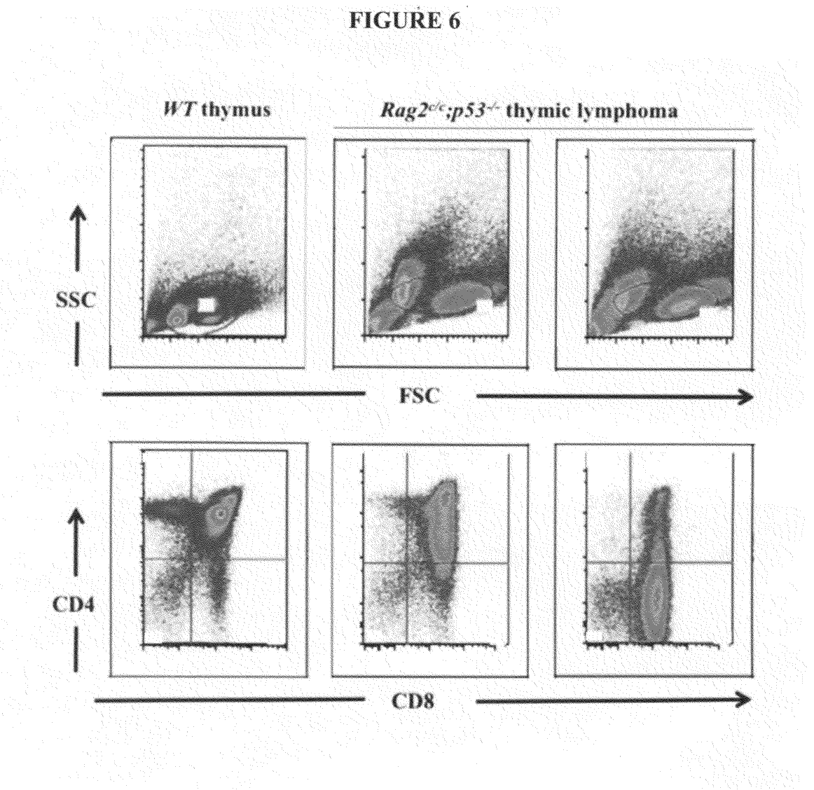

We reasoned that Rag2^{c/c} p53^{-/-} double-mutant mice might display genomic instability and lymphomagenesis, even in the context of intact classical non-homologous end joining. Consistent with previous reports (15), our Rag2^{c/c} mice displayed partial developmental blocks in B and T lymphopoiesis because of a selective V-to-DJ rearrangement defect (FIG. 5). Rag2^{c/c} animals, observed for up to 1 year, showed no obvious signs of tumorigenesis (FIG. 1A and data not shown). As expected (20), approximately two-thirds of p53^{-/-} mice developed thymic lymphoma at an average age of approximately 23 weeks (mean survival=22.8 weeks) (FIG. 1A, 1B). Similar findings in RAG/p53-deficient mice (21) demonstrate that RAG-initiated double strand breaks are not critical initiators of lymphomagenesis in p53-deficient mice. In sharp contrast, 100% (n=25) of our Rag2^{c/c} p53^{-/-} mice died within 16 weeks (mean survival=12.1 weeks) with aggressive thymic lymphomas (FIG. 1A-1C). Tumour cells were highly proliferative and expressed cell surface CD4 and CD8 (FIG. 6), with little or no surface TCR (CD3ε or TCRβ) (data not shown), indicating that these tumours originate from immature thymocytes. Tumours with highly proliferating lymphoblasts were detected in 4- to 6-week-old Rag2^{c/c} p53^{-/-} thymi, but not in other organs (data not shown), confirming their thymic origin. Rag2^{c/c} p53^{-/-} tumours generally displayed one or a few predominant Dβ1-Jβ1 or Dβ2-Jβ2 rearrangements, indicating a clonal or oligoclonal origin (FIG. 7).

We next examined genomic stability in lymphomas from Rag2^{c/c} p53^{-/-} mice, first by analysis of Giemsa-stained metaphase spreads prepared from 12 Rag2^{c/c} p53^{-/-} and two p53^{-/-} thymic lymphomas (FIG. 18). Wild-type thymocytes showed almost no abnormal metaphases (0-3%) (FIG. 18). In contrast, p53^{-/-} and Rag2^{c/c} p53^{-/-} tumours harboured a variety of cytogenetic aberrations (aberrant metaphases: 8-94%), including aneuploidy, chromosome breaks and chromosome fusions (FIG. 18). We analysed three Rag2^{c/c} p53^{-/-} thymic lymphomas using spectral (1790T and 1745T) and G-band (1779T) karyotyping (FIG. 2). We observed recurrent translocations involving chromosomes that harbour Tcr (chromosomes 14 and 6) and Ig (chromosomes 12, 6 and 16) loci, suggesting that these might have been initiated by RAG-generated breaks. Moreover, all three lymphomas harboured translocations of the Igh locus-containing chromosome 12 and/or the Tcrα/δ locus containing chromosome 14, loci that rearrange in thymocytes (22). Analysis of lymphoma 1779T revealed a C12;14 translocation (FIG. 2). These results suggest that Rag2^{c/c} p53^{-/-} T cell tumours harbour clonal translocations involving the Tcrα/δ and Igh loci, as seen in T-cell lymphomas from patients with ataxia-telangiectasia and Atm^{-/-} mice (7,8,10,11), rearrangements not observed in p53^{-/-} lymphomas (21,23).

To confirm the involvement of the Tcrα/δ locus in chromosome translocations, we performed DNA fluorescence in situ hybridization (DNA FISH) analyses on metaphases from Rag2^{c/c} p53^{-/-} thymic lymphomas (2489T and 2805T) using probes centromeric (Tcrα/δ V) and telomeric (Tcrα/δ C) to the Tcrα/δ locus plus a paint for chromosome 14 (FIG. 3A). In both tumours, breakpoints within the Tcrα/δ locus of one of the two chromosomes 14 resulted in amplification of the Tcrα/δ V region (FIG. 3A). The telomeric fragment (including Tcrα/δ C) was either translocated (2489T), or lost (2805T) (FIG. 3A). DNA FISH analysis of tumours 1790T and 1779T (from FIG. 2) using Tcrα/δ C and V probes also confirmed translocation of chromosome 14 with breakpoints within the Tcrα/δ locus, although without obvious amplification (FIG. 8).

We next performed DNA FISH on Rag2^{c/c} p53^{-/-} thymic lymphomas 2489T and 2805T using probes centromeric (Igh C) and telomeric (Igh V) to the Igh locus along with a chromosome 12 paint (FIG. 3B). In both lymphomas, one chromosome 12 showed translocation with another chromosome, with accompanying loss of both Igh C and V signals (FIG. 3B). This could result from RAG-induced breaks with loss of the telomeric end of the chromosome (including Igh V) and loss of the Igh C region by end degradation before fusion to the partner chromosome, as previously reported in Atm^{-/-} mouse T cells (8). Moreover, dual chromosome 12 and 14 paint analysis showed a C12;(14) translocation in lymphoma 2489T (FIG. 3B). In contrast to Rag2^{c/c} p53^{-/-} lymphomas, DNA FISH on metaphases from one p53^{-/-} thymic lymphoma (6960T) indicated that both Tcrα/δ and Igh loci were intact (FIG. 9), consistent with previous work (21).

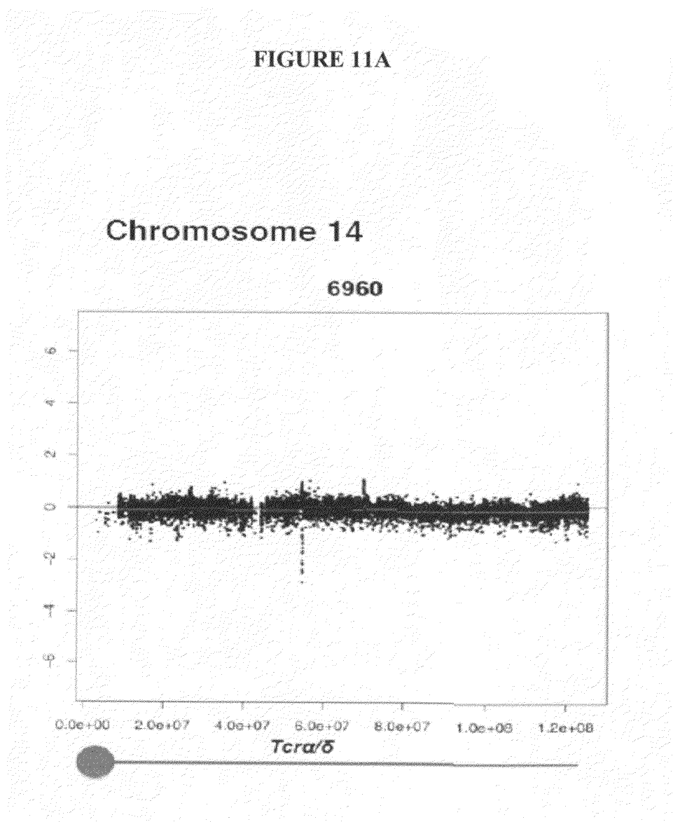

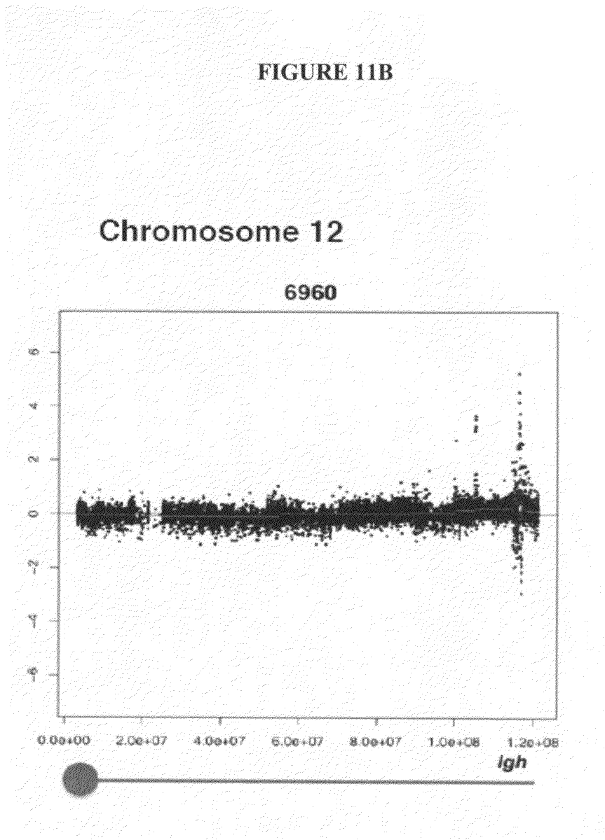

We next performed array-based comparative genomic hybridization (a-CGH) analysis on genomic DNA from five Rag2^{c/c} p53^{-/-} thymic lymphomas (2489T, 2805T, 1348T, 1779T, 1780T). We observed loss or gain of a region within the Tcrα/δ and Igh loci, reflecting V(D)J recombination (FIG. 10). All five Rag2^{c/c} p53^{-/-} lymphomas examined showed substantial amplification of a common region on chromosome 14, centromeric of the Tcrα/δ locus (FIG. 10A), in agreement with our FISH analyses (FIG. 3A). We also observed loss of a common region on chromosome 12, telomeric of the Igh locus in all five Rag2^{c/c} p53^{-/-} thymic lymphomas analysed (FIG. 10B). Tumours 1779T, 2489T and 2805 also showed loss of a large region centromeric of the Igh locus, probably reflecting DNA-end degradation before fusion to the partner chromosome (FIGS. 2 and 3A, 3B and FIG. 10B). In contrast, aCGH analysis of p53^{-/-} thymic lymphoma 6960T failed to reveal amplification centromeric to the Tcrα/δ locus or deletion telomeric to the Igh locus (FIG. 11A,11B), in agreement with our FISH analysis (FIG. 9) and previous data (23).

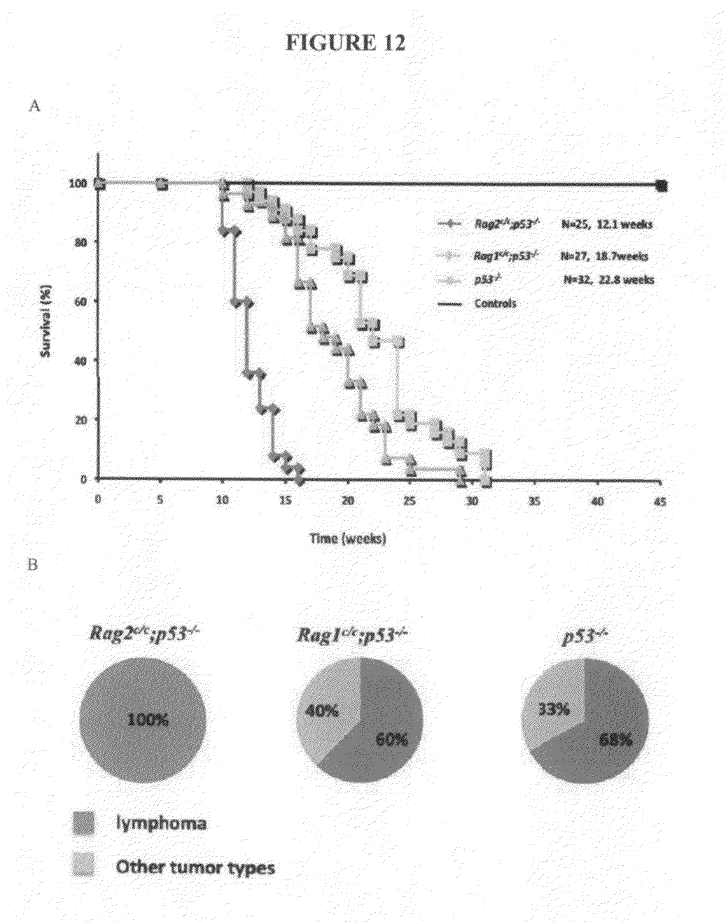

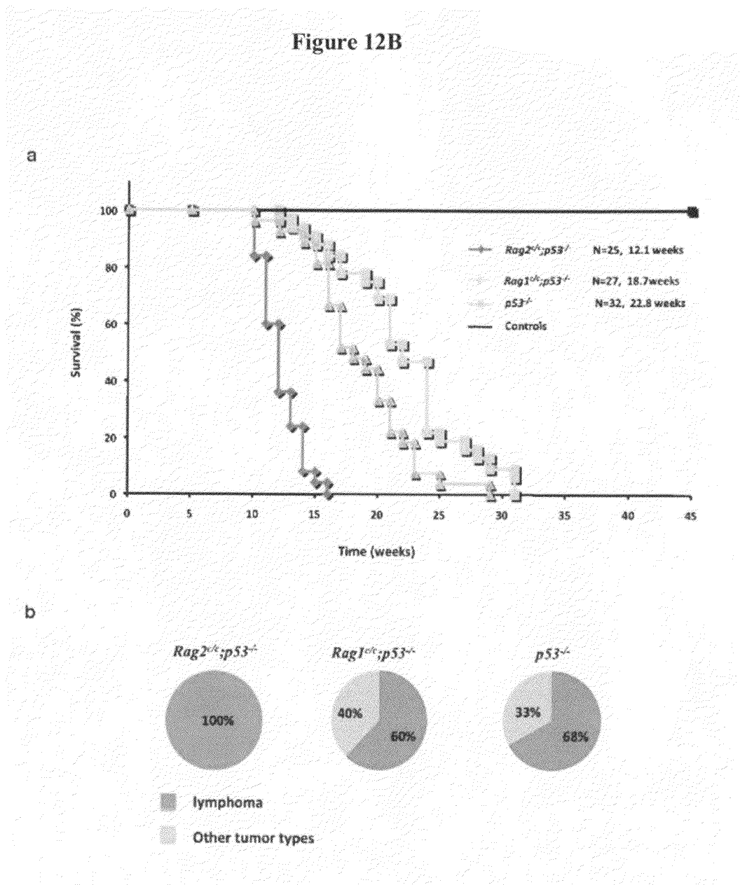

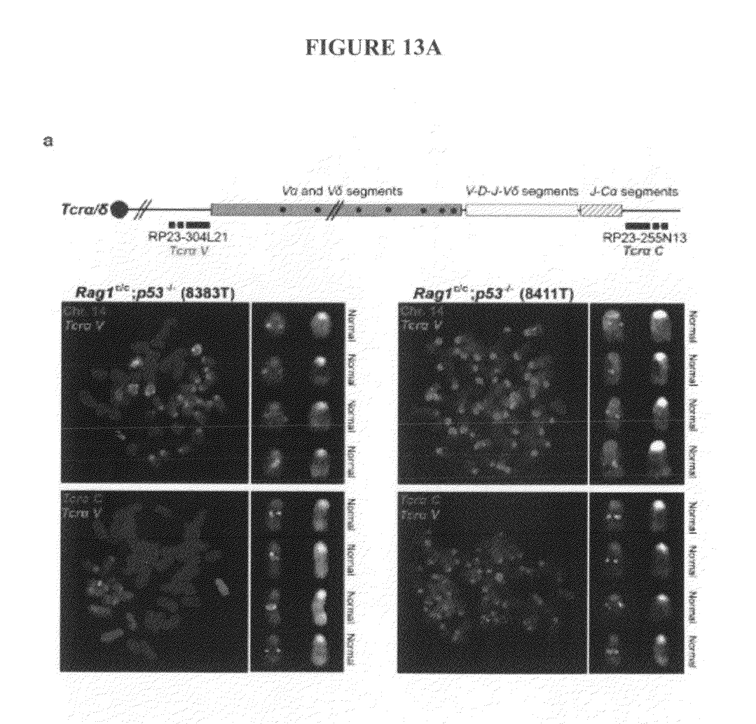

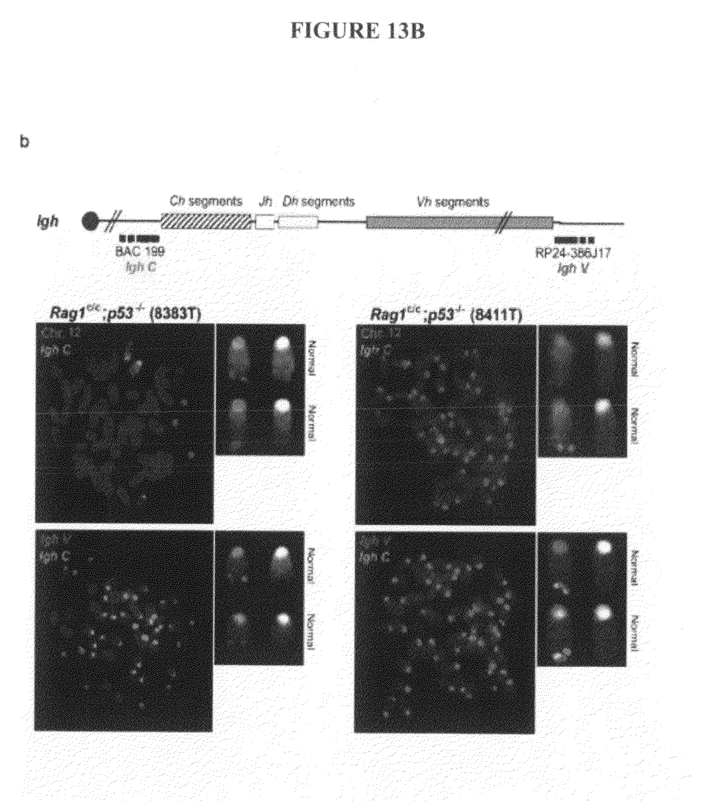

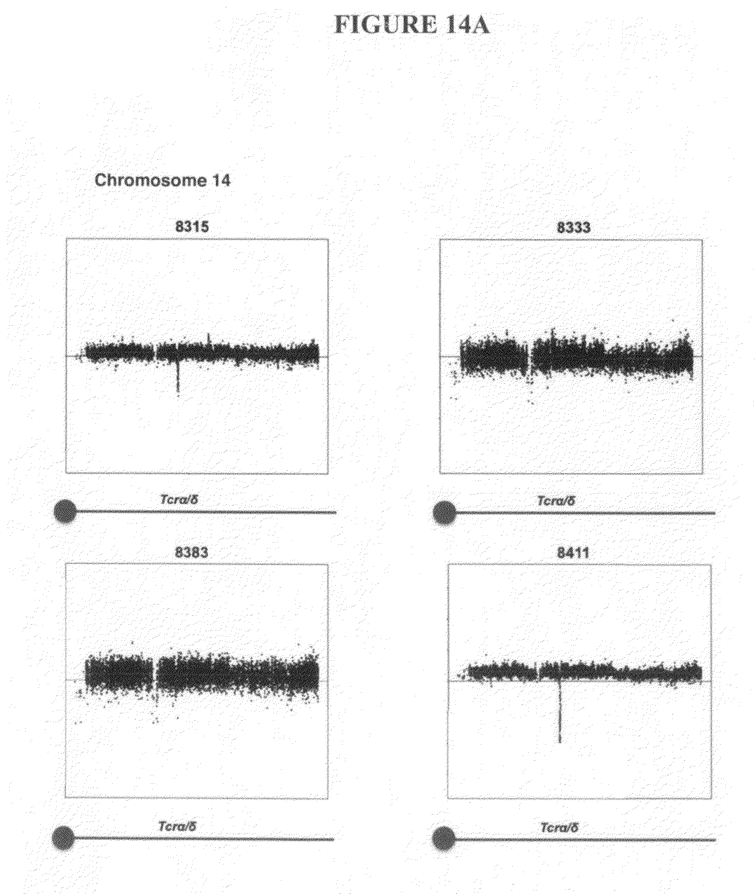

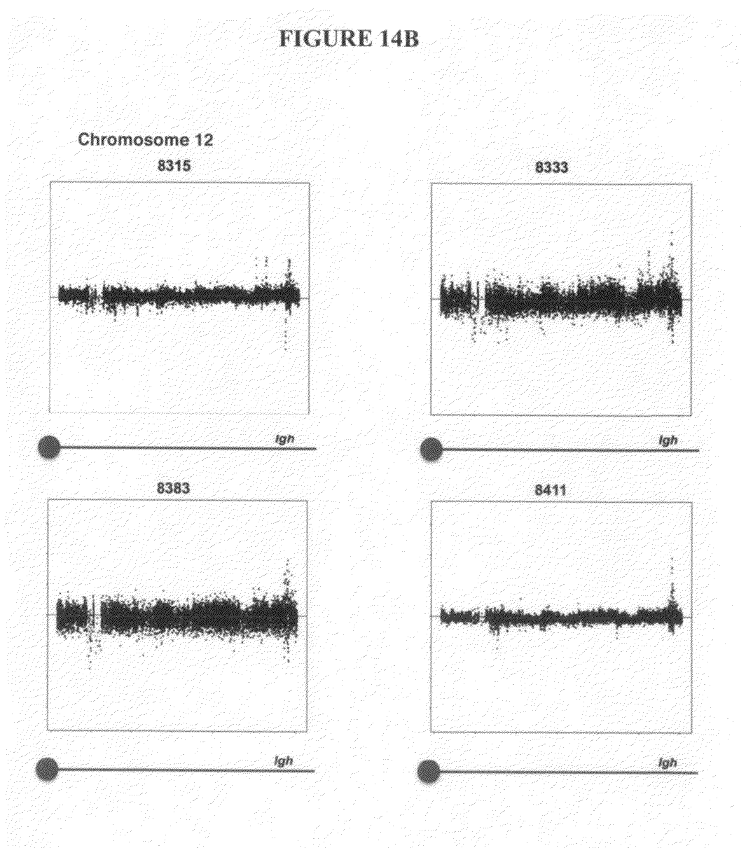

Blocking lymphocyte development in early stages can lead to persistent RAG activity, which, in the absence of p53, can provoke lymphomagenesis (23). To investigate whether the partial developmental block in Rag2^{c/c} thymocytes (15) is sufficient to produce genomic instability and lymphomagenesis, we crossed core Rag1 knock-in animals, which display diminished recombination and a strong block in B- and T-cell development (14,24) (FIG. 5), into a p53-deficient background. Rag1^{c/c} p53^{-/-} mice survived at an average age of 18.7 weeks (FIG. 12A), barely distinguishable from p53^{-/-} mice. Also like p53^{-/-} mice, only two-thirds of Rag1^{c/c} p53^{-/-} mice developed thymic lymphomas (FIG. 12B). Furthermore, metaphase DNA FISH analyses on two Rag1^{c/c} p53^{-/-} thymic lymphomas (8383T and 8411T) (FIG. 13) and a CGH analysis on genomic DNA from four Rag1^{c/c} p53^{-/-} thymic lymphomas (8315T, 8333T, 8383T, 8411T) (FIG. 14) showed no evidence of recurrent translocations, genomic amplification or genomic deletion at chromosome 14 and chromosome 12. The genomic instability observed in Rag2^{c/c} p53^{-/-} thymic lymphomas is therefore associated specifically with loss of the RAG2 C terminus, and does not result from the developmental block in core RAG2 homozygotes.

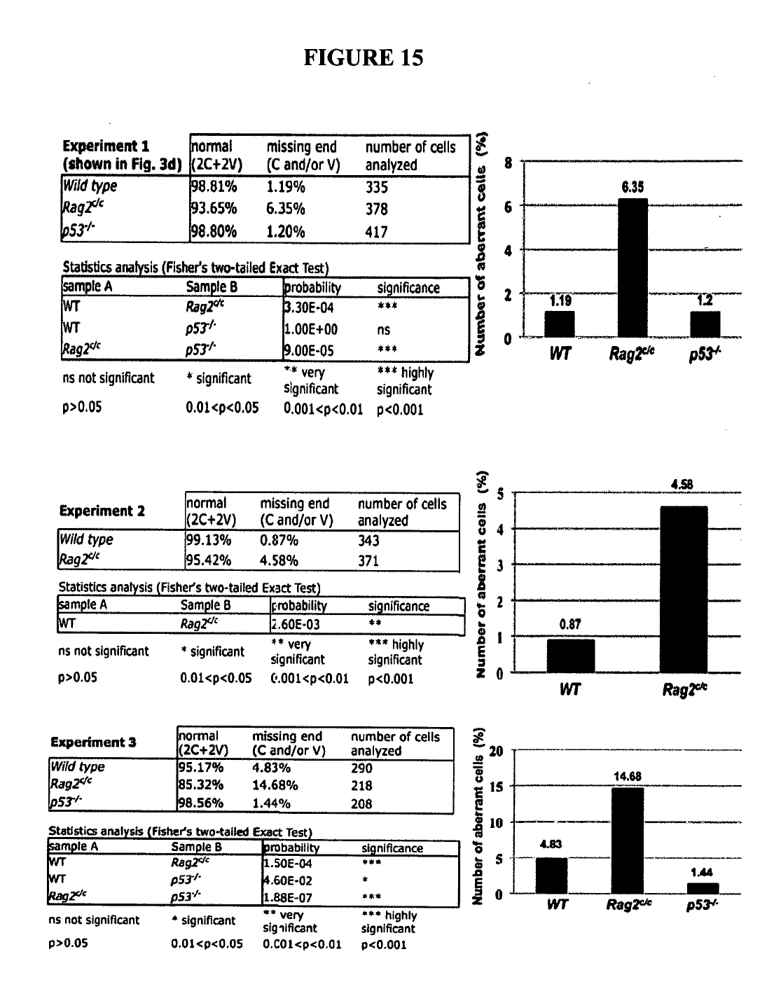

We next asked whether core RAG2 promotes genomic instability in the presence of p53 by using three-dimensional interphase DNA FISH to examine the integrity of Tcrα/δ locus (FIG. 3C) in Rag2^{c/c} double-positive thymocytes. The two alleles appeared as two pairs of signals (Tcrα/δ V and Tcra/S C, mapping the two ends of the locus) in most (>98%) wild-type and p53^{-/-} double-positive thymocytes (FIG. 3D and FIG. 15), indicating that p53 deficiency alone does not disrupt the integrity of the Tcrα/δ locus, as expected (25). In contrast, Rag2^{c/c} double-positive thymocytes displayed a three- to fivefold increase in the number of cells showing loss of at least one signal (FIG. 3C, 3D and FIG. 15). These results suggest that core RAG2 promotes genomic instability at the Tcrα/δ locus, a phenotype similar to that previously reported in Atm^{-/-} and 53bp1^{-/-} animals (9,25).

We noted that both Rag2^{c/c} p53^{-/-} and Atm^{-/-} mice feature RAG dependent genomic instability at the Tcrα/δ and Igh loci, with development of pro-T cell lymphomas bearing clonal translocations, including 12/14 translocations (2,3,7-11). To determine whether Atm^{-/-} thymic lymphomas also harbour amplification close to the Tcrα/δ locus, we performed DNA FISH analysis for Tcrα/δ and chromosome 14 on metaphases from one Atm^{-/-} thymic lymphoma (10375T) (FIG. 16A). Both chromosomes 14 showed translocations with breakpoints within the Tcrα/δ locus, and amplification of the Tcrα/δ V region on one allele (FIG. 16A), results that were confirmed by a CGH analysis (FIG. 16B). We also observed loss of DNA at a distal region of chromosome 12, near the Igh locus (FIG. 16B), as in Rag2^{c/c} p53^{-/-} lymphomas (FIG. 3A, 3B and FIG. 10). These data agree with recent analysis of thymic lymphomas from ATM-deficient mice (7). Thymic lymphomas that arise in other mutant backgrounds such as p53, core RAG1/p53 (FIGS. 12-14), Eβ/p53 or H2AX/p53 lack recurrent amplifications of chromosome 14 regions and/or recurrent chromosome 12/14 translocations, and thus appear to arise from distinct mechanisms.

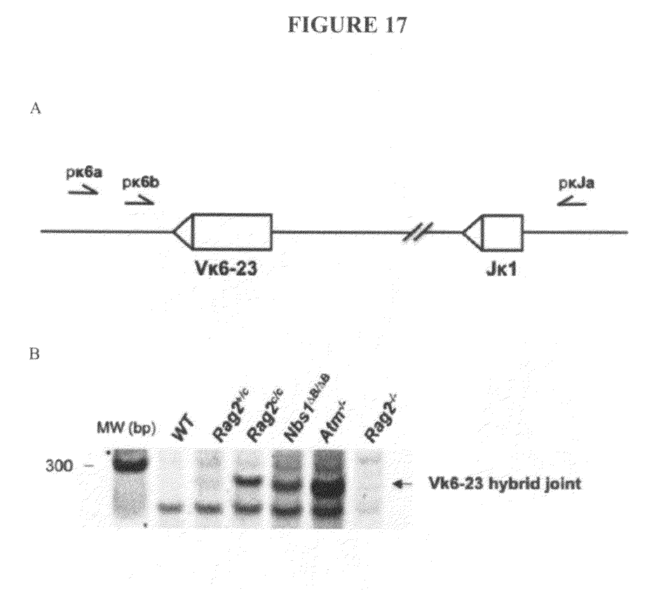

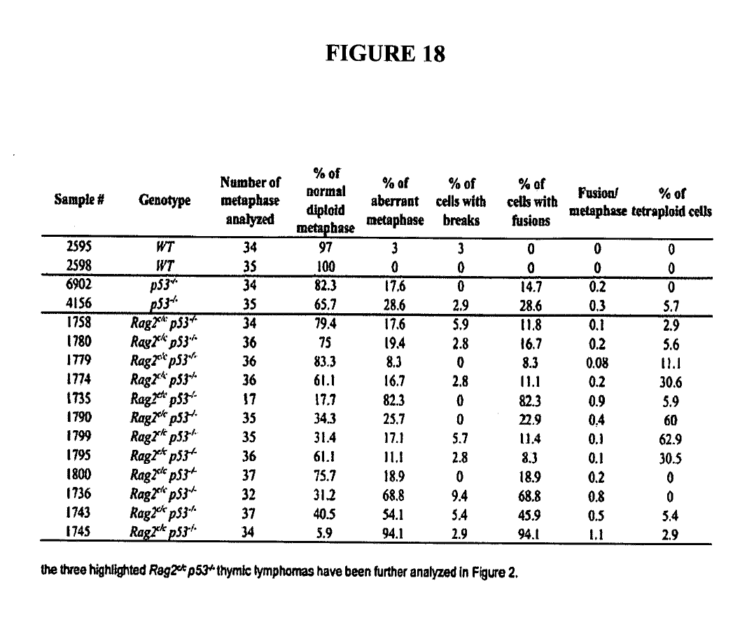

Our data reveal a novel in vivo function for the RAG2 C terminus in promoting genomic stability. How does core RAG2 allow genomic instability? We hypothesized that core RAG2, like the absence of ATM(3), destabilizes the post-cleavage complex. To investigate this, we generated RAG-signal end complexes by in vitro cleavage and challenged them at increasing temperatures, followed by gel electrophoresis (FIG. 4). Complexes containing full-length RAG2 did not release 50% of signal ends until 55° C. (FIG. 4B, 4C), as expected (13,26). In contrast, core RAG2-containing complexes displayed statistically significant instability at lower temperatures, with 50% end release at 37° C. (FIG. 4B, 4C). To examine the post-cleavage complex in vivo, we analysed inversional recombination, which requires coordination of all four DNA ends. Decreased inversional recombination and increased formation of hybrid joints (generated by joining of a coding end to a signal end, in this case revealing defects in formation of four-ended inversion products) has been reported in ATM^{-/-} and MRE11 complex deficient cells(3,27,28). As expected (3,28), we observed increased hybrid joint formation at the Igk locus (Vκ6-23 to Jκ1) in Atm^{-/-} and Nbs^{ΔB/ΔB} splenocytes (FIG. 17). Importantly, we observed increased Vκ6-23-to-Jκ1 hybrid joints in Rag2^{c/c} splenocytes, compared with their wild-type and Rag2^{c/+} counterparts (FIG. 17). These results are supported by the observation that Rag2^{c/c} lymphocytes exhibit defects in inversional recombination (15). Together, these data support our hypothesis that core RAG2 impairs the stability of the RAG post-cleavage complex in vitro and in vivo.

Our data support a common model for genomic instability in Rag2^{c/c} p53^{-/-} and Atm^{-/-} mice: premature release of RAG-generated double-strand breaks from the RAG post-cleavage complex allows ends to escape the normal joining mechanisms, to persist and to be potentially joined by alternative non-homologous end joining, a pathway permissive for chromosome translocations and amplification (4,29). Both end release and end persistence are promoted by ATM deficiency (2,3), probably because ATM both stabilizes the RAG post-cleavage complex (3) and activates p53-dependent checkpoints/apoptosis. In Rag2^{c/c} p53^{-/-} mice, end persistence might be augmented by ongoing RAG activity through the cell cycle resulting from impaired degradation of core RAG2, which lacks the cell-cycle-regulated degradation motif (18,30).

The complete penetrance, rapid development of lymphoma and extraordinary degree of RAG-mediated genomic instability make Rag2^{c/c} p53^{-/-} mice an attractive model for investigating the spectrum of somatic genome rearrangements underlying lymphomagenesis.

Methods

Mice. Mice were bred in the New York University Specific Pathogen Free facility; animal care was approved by the NYU SoM Animal Care and Use Committee (protocol number 090308-2).

Analysis of Tumour Cells. Lymphoid tumours were analysed by flow cytometry with antibodies against surface B- and T-cell markers. Metaphases were prepared and analysed as described in Methods.

FISH and Image Analysis. DNA FISH was performed using BAC probes as described in Methods. Interphase FISH was performed on double-positive thymocytes isolated by cell sorting according to protocols described in Methods. Images were obtained by confocal microscopy on a Leica SP5 AOBS system, with optical sections separated by 0.3 μm. Images were analysed using Image J software. Metaphase spreads were imaged by fluorescent microscopy on a Zeiss Imager Z2 Metasystems METAFER 3.8 system and analysed using ISIS software. Statistical analysis of image parameters used a two-tailed Fisher's exact test.

Biochemical End-Release Assay. The stability of RAG-signal end complexes was measured as described in Methods. Briefly, RAG cleavage reactions were divided into aliquots in microfuge tubes and incubated at the indicated temperatures for 30 min, followed by polyacrylamide gel electrophoresis. DNA was stained using SYBR Safe DNA Gel Stain (Invitrogen) and quantified with Quantity One software (Biorad). Student's t-test assuming equal variance was used to calculate statistical significance.

aCGH Analysis. For CGH, genomic DNA from mouse thymic lymphomas was profiled against matched thymic DNA from wild-type mice. aCGH experiments were performed on two-colour Agilent 244A Mouse Genome Microarrays. Data analysis was performed as described in Methods.

Mice. We obtained wild type (Taconic), Rag2^{c/c}(15), Rag1^{c/c}(24), p53^{-/-} (Jackson laboratory (20)) and Atm^{-/-} (Jackson laboratory (11)) mice for this study. Rag2^{c/c} or Rag1 ^{c/c} mice were bred with p53-deficient mice to generate doubly deficient mice. Genotyping of these mutants was performed by PCR of tail DNA as described in the relevant references (11,15,20,24).

Characterization of Tumour Cells and Metaphase Preparation. Lymphoid tumours were analysed by flow cytometry with antibodies against surface B-cell (CD43, B220, IgM) and T-cell (CD4, CD8, CD3, TCR-β) markers. FACS analysis used a BD LSRII flow cytometer (BD Biosciences) equipped with FacsDiVa and FlowJo. For metaphase preparation, tumour cells were prepared as previously described (31,32). Briefly, primary tumour cells were grown in complete RPMI media for 4 h and exposed to colcemid (0.04 μg/ml, GIBCO, KaryoMAX Colcemid Solution) for 2 hours at 37° C. Then, cells were incubated in KCl 75 mM for 15 min at 37 ° C., fixed in fixative solution (75% methanol/25% acetic acid) and washed three times in the fixative. Cell suspension was dropped onto pre-chilled glass slides and air-dried for further analysis.

G-Banding and Spectral Karyotyping. Optimally aged slides were treated for the induction of G-banding following the routine procedure (33). Spectral karyotyping was performed using the mouse chromosome SKY probe Applied Spectral Imaging according to the manufacturer's instructions to determine chromosomal rearrangements in the tumour samples. The slides were analysed using a Nikon Eclipse 80i microscope. G-banding as well as SKY images were captured and karyotyped using an Applied Spectral Imaging system.

DNA FISH Probes. BAC probes for the Igh and Tcrα/δ loci were labelled by nick translation and prepared as previously described (34,35). For the Igh locus, BAC 199 (Igh C) and BAC RP24-386J17 (Igh V) were labelled in Alexa Fluor 594 and 488 respectively (Molecular Probes). For the Tcrα/δ locus, BAC RP23-304L21 (Tcrα/δ V) and RP-23 255N13 (Tcrα/δ C) were labelled in Alexa Fluor 488 or 594. StarFISH-concentrated mouse FITC or Cy3 chromosome 12 or 14 paints were prepared following supplier's instructions (Cambio). BAC probes were resuspended in hybridization buffer (10% dextran sulphate, 5× Denharts solution, 50% formamide) or in paint hyb buffer, denatured for 5 min at 95° C. and pre-annealed for 45 min at 37° C. before hybridization on cells.

DNA FISH on Metaphase Spreads. Slides were dehydrated in ethanol series, denatured in 70% formamide/23 SSC (pH 7-7.4) for 1 min 30 s at 75° C., dehydrated again in cold ethanol series, and hybridized with probes o/n at 37° C. in a humid chamber. Slides were then washed twice in 50% formamide/23 SSC and twice in 23 SSC for 5 min at 37° C. each. Finally, cells were mounted in ProLong Gold (Invitrogen) containing 4′,6-diamidino-2-phenylindole (DAPI) to counterstain total DNA.

DNA FISH on Interphase Nuclei. Double-positive thymocytes were isolated from total thymi on a Beckman-Coulter MoFlo cell sorter as Thy1.2⁺ CD4⁺ CD8⁺ cells using the following antibodies: PE-Cy7-coupled anti-CD90.2 (Thy1.2; 53-2.1), APC-coupled anti-CD4 (L3T4) and FITC-coupled anti-CD8 (53-6.7). Cells were washed two times in 1×PBS and dropped onto poly-L-lysine-coated coverslips. For three-dimensional DNA FISH analyses, we used a protocol for immunofluorescence/DNA FISH previously described (34,35), with protein detection step omitted. Briefly, cells were fixed in 2% paraformaldehyde/1×PBS for 10 min at room temperature, permeabilized in 0.4% Triton/1×PBS for 5 min on ice, incubated with 0.01 mg/ml Rnase A for 1 h at 37° C. and permeabilized again in 0.7% Triton/0.1 M HCl for 10 min on ice. Cells were then denatured in 1.9 M HCl for 30 min at room temperature, rinsed in cold 1×PBS and hybridized overnight with probes at 37° C. in a humid chamber. Cells were then rinsed in 2×SSC at 37° C., 2×SCC at room temperature and 1×SSC at RT, 30 min each. Finally, cells were mounted in ProLong Gold (Invitrogen) containing DAPI to counterstain total DNA.

Biochemical End-Release Assay. End-release assay to measure the stability of the signal-end complexes was performed as previously described (26). For RAG mediated cleavage, 100 ng of recombination substrate (PCR product from p.11-1289) was incubated for 3 h at 37° C. with 200 ng purified RAG protein and 200 ng of purified recombinant HMGB1 in a buffer containing 50 mM HEPES (pH 8.0), 25 mM KCl, 4 mM NaCl, 1 mM DTT, 0.1 mg BSA, 5 mM CaCl₂ and 5 mM MgCl₂. Reactions were then divided into aliquots in microfuge tubes and incubated at different temperatures, or treated with stop buffer (10 mM Tris (pH 8.0), 10 mM EDTA, 0.2% SDS, 0.35 mg/ml proteinase K (Sigma Aldrich)) for 30 min and then run out on 4-20% acrylamide tris-borate-EDTA (TBE) gels (Invitrogen).

aCGH Analysis. aCGH experiments were performed on two-colour Agilent 244A Mouse Genome Microarray. After internal Agilent quality control, the collected data were background subtracted and normalized using the Loess method (36). We used circular binary segmentation method to define regions of copy number alteration compared with the control (37) and applied the cghMCR method for extraction of altered minimum common regions between the samples (38). The analyses and visualizations were performed using the R statistical program (39).

### REFERENCES

1. Kuppers, R. & Dalla-Favera, R. Mechanisms of chromosomal translocations in B cell lymphomas. Oncogene 20, 5580-5594 (2001).

2. Callen, E. et al. ATM prevents the persistence and propagation of chromosome breaks in lymphocytes. Cell 130, 63-75 (2007).

3. Bredemeyer, A. L. et al. ATM stabilizes DNA double-strand-break complexes during V(D)J recombination. Nature 442, 466-470 (2006).

4. Zhu, C. et al. Unrepaired DNA breaks in p53-deficient cells lead to oncogenic gene amplification subsequent to translocations. Cell 109, 811-821 (2002).

5. Gao, Y. et al. Interplay of p53 and DNA-repair protein XRCC4 in tumorigenesis, genomic stability and development. Nature 404, 897-900 (2000).

6. Difilippantonio, M. J. et al. DNA repair protein Ku80 suppresses chromosomal aberrations and malignant transformation. Nature 404, 510-514 (2000).

7. Zha, S. et al. ATM-deficient thymic lymphoma is associated with aberrant tcrd rearrangement and gene amplification. J. Exp. Med. 207, 1369-1380 (2010).

8. Callen, E. et al. Chimeric IgH-TCRa/d translocations in T lymphocytes mediated by RAG. Cell Cycle 8, 2408-2412 (2009).

9. Matei, I. R. et al. ATMdeficiency disrupts Tcra locus integrity and the maturation of CD41CD81 thymocytes. Blood 109, 1887-1896 (2007).

10. Liyanage, M. et al. Abnormal rearrangement within the a/d T-cell receptor locus in lymphomas from Atm-deficient mice. Blood 96, 1940-1946 (2000).

11. Barlow, C. et al. Atm-deficient mice: a paradigm of ataxia telangiectasia. Cell 86, 159-171 (1996).

12. Corneo, B. et al. Rag mutations reveal robust alternative end joining. Nature 449, 483-486 (2007).

13. Lee, G. S., Neiditch, M. B., Salus, S. S. & Roth, D. B. RAG proteins shepherd double strand breaks to a specific pathway, suppressing error-prone repair, but RAG nicking initiates homologous recombination. Cell 117, 171-184 (2004).

14. Jones, J. M. & Simkus, C. The roles of the RAG1 and RAG2 “non-core” regions in V(D)J recombination and lymphocyte development. Arch. Immunol. Ther. Exp. (Warsz.) 57, 105-116 (2009).

15. Liang, H. E. et al. The “dispensable” portion of RAG2 is necessary for efficient V-to-DJ rearrangement during B and Tcell development. Immunity 17,639-651(2002).

16. Qiu, J. X., Kale, S. B., Yarnell Schultz, H. & Roth, D. B. Separation-of-function mutants reveal critical roles for RAG2 in both the cleavage and joining steps of V(D)J recombination. Mol. Cell 7, 77-87 (2001).

17. Steen, S. B., Han, J.-O., Mundy, C., Oettinger, M. A. & Roth, D. B. Roles of the “dispensable” portions ofRAG-1 andRAG-2 in V(D)J recombination. Mol. Cell. Biol. 19, 3010-3017 (1999).

18. Curry, J. D. & Schlissel, M. S. RAG2′s non-core domain contributes to the ordered regulation of V(D)J recombination. Nucleic Acids Res. 36, 5750-5762 (2008).

19. Talukder, S. R., Dudley, D. D., Alt, F. W., Takahama, Y. & Akamatsu, Y. Increased frequency of aberrant V(D)J recombination products in core RAG-expressing mice. Nucleic Acids Res. 32, 4539-4549 (2004).

20. Jacks, T. et al. Tumor spectrum analysis in p53-mutant mice. Curr. Biol. 4, 1-7 (1994).

21. Liao, M. J. et al. No requirement for V(D)J recombination in p53-deficient thymic lymphoma. Mol. Cell. Biol. 18, 3495-3501 (1998).

22. Forster, A., Hobart, M., Hengartner, H. & Rabbitts, T. H. An immunoglobulin heavychain gene is altered in two T-cell clones. Nature 286, 897-899 (1980).

23. Haines, B. B. et al. Block of T cell development in P53-deficient mice accelerates development of lymphomas with characteristic RAG-dependent cytogenetic alterations. Cancer Cell 9, 109-120 (2006).

24. Dudley, D. D. et al. Impaired V(D)J recombination and lymphocyte development in core RAG1-expressing mice. J. Exp. Med. 198, 1439-1450 (2003).

25. Difilippantonio, S. et al. 53BP1facilitates long-rangeDNA end-joining during V(D)J recombination. Nature 456, 529-533 (2008).

26. Arnal, S. M., Holub, A. J., Salus, S. S. & Roth, D. B. Non-consensus heptamer sequences destabilize the RAG post-cleavage complex, making ends available to alternative DNA repair pathways. Nucleic Acids Res. 38, 2944-2954 (2010).

27. Helmink, B. A. et al. MRN complex function in the repair of chromosomal Rag mediated DNA double-strand breaks. J. Exp. Med. 206, 669-679 (2009).

28. Deriano, L., Stracker, T. H., Baker, A., Petrini, J. H. & Roth, D. B. Roles for NBS1 in alternative nonhomologous end-joining of V(D)J recombination intermediates. Mol. Cell 34, 13-25 (2009).

29. Simsek, D. & Jasin, M. Alternative end-joining is suppressed by the canonical NHEJ component Xrcc4-ligase IV during chromosomal translocation formation. Nature Struct. Mol. Biol. 17, 410-416 (2010).

30. Li, Z., Dordai, D. I., Lee, J. & Desiderio, S. A conserved degradation signal regulates RAG-2 accumulation during cell division and links V(D)J recombination to the cell cycle. Immunity 5, 575-589 (1996).

31. Theunissen, J. W. & Petrini, J. H. Methods for studying the cellular response toDNA damage: influence of the Mre11 complex on chromosome metabolism. Methods Enzymol. 409, 251-284 (2006).

32. Multani, A. S. et al. Caspase-dependent apoptosis induced by telomere cleavage and TRF2 loss. Neoplasia 2, 339-345 (2000).

33. Pathak, S. Chromosome banding techniques. J. Reprod. Med. 17, 25-28 (1976).

34. Hewitt, S. L. et al. RAG-1 and ATM coordinate monoallelic recombination and nuclear positioning of immunoglobulin loci. Nature Immunol. 10, 655-664(2009).

35. Skok, J. A. et al. Reversible contraction by looping of the Tcra and Tcrb loci in rearranging thymocytes. Nature Immunol. 8, 378-387 (2007).

36. Yang, Y. H. et al. Normalization for cDNA microarray data: a robust composite method addressing single and multiple slide systematic variation. Nucleic Acids Res. 30, el5 (2002).

37. Olshen, A. B., Venkatraman, E. S., Lucito, R. & Wigler, M. Circular binary segmentation for the analysis of array-based DNA copy number data. Biostatistics 5, 557-572 (2004).

38. Aguirre, A. J. et al. High-resolution characterization of the pancreatic adenocarcinoma genome. Proc. Natl Acad. Sci. USA 101, 9067-9072 (2004).

39. R. development Core Team. R: A Language and Environment for Statistical Computing. Vienna: R Foundation for Statistical Computing (2006).

40. Gartner, F. et al. Immature thymocytes emply distance signaling pathways for allelic exclusion versus differentiation and expansion. Immunity 10, 537-546, doi:S1074-7613(00)80053-9 [pii] (1999).

41. Mills, K. D., Ferguson, D. O., & Alt, F. W., The role of DNA breaks in genomic instability and tumorigenesis. Immunol Rev 194, 77-95 (2003).

42. Akamatsu, Y. et al., Deletion of the RAG2 C terminus leads to impaired lymphoid development in mice. Proc Natl Acad Sci USA 100 (3), 1209-1214 (2003).

43. Curry, J. D. et al., Chromosomal reinsertion of broken RSS ends during T cell development. J Exp Med 204 (10), 2293-2303 (2007).

44. Mullighan, C. Downing J. Global genomic characterization of acute lymphoblastic leukemia. Seminars in Hematology 46:3- (2009).

45. Jankovic, M., Nussenzweig, A., & Nussenzweig, M. C., Antigen receptor diversification and chromosome translocations. Nat Immunol 8 (8), 801-808 (2007).

46. Agrawal, A. & Schatz, D. G., RAG1 and RAG2 form a stable postcleavage synaptic complex with DNA containing signal ends in V(D)J recombination. Cell 89, 43-53 (1997).

47. Nacht, M. & Jacks, T., V(D)J recombination is not required for the development of lymphoma in p53-deficient mice. Cell Growth Differ 9 (2), 131-138 (1998).

48. Born, W., White, J., Kappler, J., & Marrack, P., Rearrangement of IgH genes in normal thymocyte development. J Immunol 140 (9), 3228-3232 (1988).

49. Taylor, A. M., Metcalfe, J. A., Thick, J., & Mak, Y. F., Leukemia and lymphoma in ataxia telangiectasia. Blood 87 (2), 423-438 (1996).

This invention may be embodied in other forms or carried out in other ways without departing from the spirit or essential characteristics thereof. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

What is claimed is:

1. A transgenic mouse whose genome comprises a C-terminal truncation of RAG2 and the loss of p53, wherein said mouse is capable of the rapid development of T-cell lymphoma.

2. The transgenic mouse of claim 1 wherein the C-terminal truncation of RAG2 results in RAG2 protein having only amino acids corresponding to amino acids 1-383 of mouse RAG2.

3. A non-human transgenic animal model for hematologic malignancies caused by chromosomal translocations wherein the animal lacks p53 and its genome comprises a modified version of a gene encoding recombination activating gene 2 (RAG2) wherein the encoded RAG2 protein lacks a C-terminal region.

4. The animal model of claim 3 wherein the encoded RAG2 protein lacks amino acids corresponding to amino acids 384-526 in mouse RAG2.

5. The animal model of claim 3 wherein the encoded RAG2 protein has only amino acids corresponding to amino acids 1-383 of mouse RAG2.

6. The animal model of claim 3 wherein the hematological malignancy is T-cell acute lymphoblastic leukemia (T-ALL).

7. A non-human transgenic animal which is mutant for p53 and RAG2 whose genome comprises a modified version of a gene encoding p53 whereby p53 is lacking in the animal and whose genome also comprises a modified version of a gene encoding RAG2 wherein the encoded RAG2 protein lacks a C-terminal region.

8. The non-human transgenic animal of claim 7, wherein the encoded RAG2 protein lacks amino acids corresponding to amino acids 384-526 in mouse RAG2.

9. The animal model of claim 3 or the non-human transgenic animal of claim 7, wherein said animal is a mouse.

10. A method of screening test drugs or agents that inhibit or suppress T-cell acute lymphoblastic leukemia, comprising: contacting or otherwise exposing the transgenic mouse of claim 1 to a test drug or agent, wherein expression in T-lymphocytes rapidly induces T-cell acute lymphoblastic leukemia; comparing the leukemia in said transgenic mouse after contact or exposure to said test drug or agent relative to the leukemia of said mouse prior to contact or exposure with said test drug or agent; wherein suppression of the leukemia in said transgenic mouse after contact or exposure to said test drug or agent relative to the leukemia of said mouse prior to contact or exposure with said test drug or agent is indicative of a test drug or agent that suppresses T-cell acute lymphoblastic leukemia.

11. A method for screening a candidate compound for modulation of hematological malignancy comprising: (a) administering the candidate compound to the non-human transgenic animal of claim 3 or to cells or a cell line derived from the non-human transgenic animal of claim 3; and (b) monitoring an effect of said compound on the non-human transgenic animal or on the cells or cell line.

12. The method of claim 11, wherein monitoring the effect comprises detecting the levels of abnormal T cells in the animal or the cells of the cell line.

13. The method of claim 11, wherein monitoring the effect comprises detecting the levels of chromosomal translocations in the animal, in the cells of the animal, or the cells of the cell line.

14. The method of claim 11, wherein monitoring the effect comprises detecting levels of genetic instability at the Tcrα/δ and Igh loci.

15. A method of screening agents potentially useful for treating, preventing or inhibiting T-cell lymphoma, comprising: a) administering an agent to a first transgenic animal according to claim 7; b) observing the ability of the first transgenic animal to develop T-cell lymphoma; and c) comparing the ability of the first transgenic animal to develop T-cell lymphoma to the ability of a second transgenic animal according to claim 7 to develop T-cell lymphoma, the agent not being administered to the second transgenic animal; wherein a decrease in development of T-cell lymphoma in the first transgenic animal indicates that the agent is potentially useful for treating, preventing or inhibiting T-cell lymphoma.

16. A cell or cell line derived from the non-human transgenic animal of claim 1 or 5.
